(19) United States
(12) Patent Application Publication
(10) Pub. No.: US 2017/0290846 A1
(43) Pub. Date: Oct. 12, 2017

(54) IN SITU GELLING FORM FOR LONG-ACTING DRUG DELIVERY

(71) Applicant: University of Tennessee Research Foundation, Memphis, TN (US)

(72) Inventors: Tao L. Lowe, Germantown, TN (US); James Johnson, Memphis, TN (US); Linfeng Wu, Memphis, TN (US)

(21) Appl. No.: 15/512,439

(22) PCT Filed: Sep. 30, 2015
(86) PCT No.: PCT/US15/53205

§ 371 Date: Mar. 17, 2017

Related U.S. Application Data
(60) Provisional application No. 62/057,510, filed on Sep. 30, 2014.

(51) Int. Cl. A61K 31/567; A61K 47/22; A61K 47/34; A61K 9/00

(52) U.S. Cl. CPC A61K 31/567; A61K 9/0024; A61K 47/22; A61K 47/34

(57) ABSTRACT

The present invention relates to an injectable polymer matrix drug delivery system comprising a biodegradable polymer, a solvent or a combination of solvents, and an active pharmaceutical ingredient.

### RELATED APPLICATION

This application claims priority to U.S. Provisional Application No. 62/057,510, filed Sep. 30, 2014, the entire contents of which are incorporated herein by reference.

### BACKGROUND

In situ controlled-release drug delivery offers many advantages and avoids certain disadvantages associated with traditional drug delivery methods. For example, in situ rate-controlled drug administration avoids the variability in absorption and metabolism associated with oral therapy. It further provides continuity of drug administration, permitting the use of a pharmacologically active agent with a short biological half-life. Moreover, there is less chance of over- or under-dosing with an in situ drug delivery regimen, and patient compliance with a multi-day, -week, or -month in situ drug delivery regimen is superior to frequent oral dosing.

However, drug-delivery systems using most of the known biodegradable polymers have been rigid materials. In such instances, the drug is incorporated into the polymer, and the mixture is shaped into a certain form, such as a cylinder, disc, or fiber for implantation.

Further, in the process of preparing rigid drug delivery systems, biologically active substances are commonly exposed to extreme stresses. Necessary manufacturing steps may include excessive exposure to heat, pH extremes, cross-linking agents, freezing, and drying. Following manufacture or preparation, the drug delivery systems must be stored for some extended period of time prior to administration, and little information is available on the subject of long term stability of therapeutics within solid biodegradable delivery systems.

Rigid polymers can be inserted into the body with a syringe or catheter in the form of small particles, such as microspheres or microcapsules. However, because they are still solid particles, they do not form the continuous and nearly homogeneous, monolithic matrix that is sometimes needed for preferred release profiles.

In addition, microspheres or microcapsules prepared from these polymers and containing biologically active substances to be released into the body are sometimes difficult to produce on a large scale. Most of the microencapsulation processes involve high temperature and contact with organic solvents, steps that tend to damage the bioactivity of therapeutics. Moreover, their storage often presents problems and, upon injection, their granular nature can cause blockages in injection devices or irritation of the soft tissues into which the small particles are injected.

Thus, there exists a need for a composition and method for providing a flexible or flowable biodegradable composition that can form a gel in situ and be used in vivo to release a variety of different biologically active substances. There is also a continuing need for biodegradable polymer compositions that may provide controlled release in such a way that trauma to the surrounding soft tissues can be minimized.

### SUMMARY

Provided herein is a drug delivery system, and methods for using the drug delivery system to deliver an active pharmaceutical ingredient to a subject in need thereof.

In one aspect, provided herein is an injectable polymer matrix drug delivery system comprising: a) a biodegradable polymer or combinations thereof; b) a solvent or a combination of solvents; and c) an active pharmaceutical ingredient. In one embodiment the active pharmaceutical ingredient is a birth control agent.

In one embodiment, provided herein is a method of inducing amenorrhea, the method comprising administering to a subject in need thereof the injectable polymer matrix drug delivery system described herein.

In still another embodiment, provided herein is a method of reducing or inhibiting spermatogenesis, the method comprising administering to a subject in need thereof the injectable polymer matrix drug delivery system described herein.

In yet another embodiment, provided herein is a method of minimizing uterine bleeding, the method comprising administering to a subject in need thereof the injectable polymer matrix drug delivery system described herein.

In another embodiment, provided herein is a method of minimizing estrus, the method comprising administering to a subject in need thereof the injectable polymer matrix drug delivery system described herein.

In still another embodiment, provided herein is a method of forming a polymer matrix drug delivery system described herein comprising: a) adding an active pharmaceutical ingredient to a solvent or a combination of solvents; b) dissolving or dispersing the active pharmaceutical ingredient; c) adding the dissolved or dispersed active pharmaceutical solution to a biodegradable polymer or combinations thereof; and d) mixing the dissolved or dispersed active pharmaceutical ingredient and biodegradable polymer solution to homogeneity; such that the polymer matrix drug delivery system is formed.

### BRIEF DESCRIPTION OF THE DRAWINGS

FIG. 1 shows the viscosity of polymer solutions described herein.

FIG. 2A shows the in vitro cumulative release of levonorgestrel (LNG) from formulations 55, 56, 57, 61, 62, 63, 64, and 67 as a % cumulative amount.

FIG. 2B shows the in vitro cumulative release of LNG from formulations 55, 64, and 96 as a % cumulative release.

FIG. 3A shows the in vitro daily release of LNG from formulations 55, 64 (tamarin) and 96 as a function of amount released per day.

FIG. 3B shows the in vitro daily release of LNG from formulations 55, 64 (tamarin) and 96 as a function of percent cumulative release.

FIG. 4A shows that LNG is continuously slowly released from each of the night formulations with above 4 μg per day for two months in formulations 96-100.

FIG. 4B shows that LNG is continuously slowly released from each of the night formulations with above 4 μg per day for two months in formulations 101-105.

FIG. 5A shows that LNG is continuously slowly released from each of the night formulations with above 4 μg per day for two months in formulations 96-100.

FIG. 5B shows that LNG is continuously slowly released from each of the night formulations with above 4 μg per day for two months in formulations 101-105.

FIG. 6A shows the LNG release profiles from the tamarin formulation in two different volumes 160 and 400 μL in micrograms cumulative LNG released.

FIG. 6B shows the LNG release profiles from the tamarin formulation in two different volumes 160 and 400 μL in % cumulative LNG released.

FIG. 6C shows the LNG release profiles from the tamarin formulation in two different volumes 160 and 400 μL in micrograms LNG released daily.

FIG. 7 shows the LNG Plasma concentration as a function of time after sub-Q injection of the 64 (tamarin), 55 and 96 formulations containing 10 mg LNG in female rat. Each point represents the mean±SE, n=8.

FIG. 8A shows individual LNG Plasma concentrations against time from tamarin formulation.

FIG. 8B shows individual LNG Plasma concentrations against time from formulation 96.

FIG. 8C shows individual LNG Plasma concentrations against time from formulation 55.

FIG. 9A shows changes in body weights of animals subcutaneously injected with tamarin, 55-LNG, 96-LNG, 55-LNG-B formulations and control animals.

FIG. 9B shows changes in body weights of animals subcutaneously injected with tamarin (64), 55-LNG and 96-LNG formulations and control animals.

FIG. 10 displays the vaginal cytology after injection of the tamarin formulation.

FIG. 11 displays the vaginal cytology after injection of the LNG (55) formulation.

FIG. 12 displays the vaginal cytology after injection of the LNG (96) formulation.

FIG. 13 shows LNG Plasma concentration as a function of time after sub-Q injection of tamarin-LNG formulation. Each point represents the mean ±SE, n=5.

FIG. 14 shows LNG Plasma concentration measured in individual rats as a function of time after sub-Q injection of tamarin-LNG formulation (40 mg·kg-1).

FIG. 15 shows body weights of rats subcutaneously injected with tamarin-LNG formulation. Each point represents the mean ±SE, n=5.

FIG. 16 shows a standard graph for LNG spiked together in blank rat plasma.

FIG. 17 shows a standard graph for LNG-B spiked together in blank rat plasma. FIG. 18 shows in vitro release of 64 and 96 formulations in the E and F release medium conditions.

FIG. 19 shows LNG accumulated release percentage as a function of time in 96.a, 96.b and 96.c formulations containing NMP/BB as a solvent system. Each point represents the mean ±SE, n=3.

FIG. 20 shows LNG accumulated release percentage as a function of time in 96.d, 96.e and 96.f formulations containing NMP/BB as a solvent system. Each point represents the mean ±SE, n=3.

FIG. 21 shows LNG accumulated release percentage as a function of time in 96.g, 96.h and 96.i formulations containing NMP/BB as a solvent system. Each point represents the mean ±SE, n=3.

FIG. 22 shows LNG accumulated release percentage as a function of time in 96.j, 96.k and 96.l formulations containing NMP/TEC as a solvent system. Each point represents the mean ±SE, n=3.

FIG. 23 shows LNG accumulated release percentage as a function of time in 96.m, 96.n and 96.o formulations containing NMP/TEC as a solvent system. Each point represents the mean ±SE, n=3.

FIG. 24 shows LNG accumulated release percentage as a function of time in Tamarin and diluted Tamarin formulations containing NMP/TEC as a solvent system. Each point represents the mean ±SE, n=3.

FIG. 25A and FIG. 25B shows LNG accumulated release percentage as a function of time for 24 hours in different formulations. Each point represents the mean.

FIG. 25C and FIG. 25D shows LNG accumulated release percentage as a function of time for 14 days in different formulations. Each point represents the mean.

FIG. 26 shows the shear viscosity of different formulations that were tested for accelerated release studies. Each point represents the mean ±SE, n=3.

FIG. 27 shows LNG Plasma concentration as a function of time after sub-Q injection of 96.r formulation. Each point represents the mean ±SE, n=3.

FIG. 28 shows LNG Plasma concentration measured in individual rats as a function of time after sub-Q injection of 96.r formulation (40 mg·kg−1.

FIG. 29 shows changes in body weight of rats subcutaneously injected with 96.r formulation.

FIG. 30 shows LNG Plasma concentration as a function of time after sub-Q injection of 96.zz formulation. Each point represents the mean ±SE, n=3.

FIG. 31 shows LNG Plasma concentration measured in individual rats as a function of time after sub-Q injection of 96.zz formulation (40 mg·kg−1.

FIG. 32 shows changes in body weight of rats subcutaneously injected with 96.zz formulation.

FIG. 33 shows LNG plasma concentration as a function of time after sub-Q injection of 64.a formulation. Each point represents the mean ±SE, n=3.

FIG. 34 shows LNG Plasma concentration measured in individual rats as a function of time after sub-Q injection of 64.a formulation (40 mg·kg−1).

FIG. 35 shows changes in body weight of rats subcutaneously injected with 64.a formulation.

FIG. 36 shows the representative pressure required to expel selected formulations as a function of extruded volume (mL) at the crosshead speed of 1 mm/s.

FIG. 37 shows the activation force required for formulations injected into a vial by texture analyzer. The values are expressed as the mean of three determinations.

FIG. 38 shows the average force required for formulations injected into a vial by texture analyzer. The values are expressed as the mean of three determinations.

### DETAILED DESCRIPTION

Provided herein is an injectable drug delivery system, comprising a biodegradable polymer, a solvent, or combination of solvents, and an active pharmaceutical ingredient. Also provided herein are methods related to administration of the injectable drug delivery system described herein.

The biodegradable polyesters in the formulations used in the drug delivery system will gradually completely degrade on site after injection. Owing to the complete degradation of the delivery system, no polymers will accumulate in the body and no surgical removal will be required. The drug delivery system can be used by lower-level health care providers and may even be self-administered by subjects, in need thereof, by simple subcutaneous or intramuscular injection.

In one aspect, the drug delivery system described herein is an injectable polymer matrix drug delivery system comprising:

a) a biodegradable polymer selected from the group consisting of polyester, poly(lactic-co-glycolic acid), poly(lactic acid), poly(ε-caprolactone), poly(ethylene glycol-block-lactic acid), poly(alkylcyanoacrylate), polyanhydride, poly(bis(p-carboxyphenoxy) propane-sebacic acid), polyorthoester, polyphosphoester, polyphosphazene, polyurethane, and poly(amino acid), or combinations thereof;

b) a solvent or a combination of solvents; and

c) an active pharmaceutical ingredient.

In one embodiment, the biodegradable polymer is selected from the group consisting of polyester, poly(lactic-co-glycolic acid) (PLGA), poly(lactic acid), poly(ε-caprolactone), poly(ethylene glycol-block-lactic acid), poly(alkylcyanoacrylate), polyanhydride, poly(bis(p-carboxyphenoxy) propane-sebacic acid), polyorthoester, polyphosphoester, polyphosphazene, polyurethane, and poly(amino acid), or combinations thereof among themselves or their copolymers and/or blends with poly(ethylene glycol) (PEG).

The biodegradable polymer may be PLGA in some embodiments. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid-glycolic acid ratio. In some embodiments, PLGA to be used in accordance with the methods and systems described herein is characterized by a lactic acid:glycolic acid ratio of approximately 85:15, approximately 75:25, approximately 60:40, approximately 50:50, approximately 40:60, approximately 25:75, or approximately 15:85.

Similarly, the degradation rate of combinations of biodegradable polymers can be adjusted by altering the relative ratios of the polymers. Thus, in some embodiments, combinations to be used in accordance with the methods and systems described herein are characterized by ratios of approximately 85:15, approximately 75:25, approximately 60:40, approximately 50:50, approximately 40:60, approximately 25:75, or approximately 15:85. For example, provided herein are combinations of poly(lactic acid) and poly(lactic-co-glycolic acid) in ratios of approximately 5:1, 4:1, and 1:1.

In one embodiment, the biodegradable polymer of the drug delivery system described herein is selected from poly(lactic-co-glycolic acid), poly(lactic acid), and poly(ε-caprolactone), or combinations thereof.

In another embodiment, the biodegradable polymer of the drug delivery system described herein is selected from poly(L-lactic acid) and poly(D,L-lactic acid), or combinations thereof.

In still another embodiment, the drug delivery system described herein comprises the polymer in about 0-50% by weight, the solvent in about 50-95% by weight, and the pharmaceutical ingredient in about 0.1-30% by weight.

### Solvents

As used herein, the term “solvent” refers to an organic compound capable of dissolving a solute. Solvents described herein may be non-polar, semi-non-polar, semi-polar, or polar. In preferred embodiments described herein, the solvents are semi-non-polar, semi-polar, or polar. In other preferred embodiments described herein, the solvents are semi-polar or polar. One example of a non-polar solvent is pentane, and one example of a polar solvent is water.

In one embodiment, the solvent of the drug delivery system described herein is selected from N-methyl-2-pyrrolidone (NMP), benzyl benzoate (BB), benzyl alcohol (BA), triethyl citrate (TEC), acetyl triethyl citrate (ATEC), ethyl acetate (EA), and acetyl tributyl citrate (ATBC), or combinations thereof.

In another embodiment, the solvent of the drug delivery system described herein is selected from N-methyl-2-pyrrolidone (NMP), benzyl benzoate (BB), benzyl alcohol (BA), triethyl citrate (TEC), acetyl triethyl citrate (ATEC), and ethyl acetate (EA), or combinations thereof.

In yet another embodiment, the solvent combination of the drug delivery system described herein is NMP and TEC; NMP and ATEC; NMP and ATBC; NMP and BB; NMP and BA; NMP and EA; TEC and BB; ATEC and BB; ATBC and BB; TEC and BA; ATEC and BA; ATBC and BA; TEC and EA; ATEC and EA; ATBC and EA; NMP, TEC and BB; NMP, ATEC and BB; NMP, ATBC and BB; NMP, TEC and BA; NMP, ATEC and BA; NMP, ATBC and BA; NMP, TEC and EA; NMP, ATEC and EA; NMP, ATBC and EA; TEC, BB and EA; ATEC, BB and EA; ATBC, BB and EA; TEC, BA and EA; ATEC, BA and EA; or ATBC, BA and EA.

In still another embodiment, the solvent combination of the drug delivery system described herein is NMP and TEC, NMP and ATEC, NMP and BB, NMP and BA, or NMP and EA.

In other embodiments, the solvent combination of the drug delivery system described herein is NMP and TEC, NMP and ATEC, NMP and BB, or NMP and BA.

### Active Pharmaceutical Ingredient

One of the components of the drug delivery system described herein is an active pharmaceutical ingredient. As used herein, the term “active pharmaceutical ingredient” refers to a substance intended to furnish pharmacological activity or to otherwise have direct effect in the diagnosis, cure, mitigation, treatment or prevention of disease, or to have direct effect in restoring, correcting or modifying physiological functions in a subject. By varying the drug's administered dosage, the effect in a subject may vary. Some drugs may comprise more than one kind of active pharmaceutical ingredient.

In one embodiment, the active pharmaceutical ingredient of the drug delivery system described herein is an anti-inflammatory agent, an antibacterial agent, an antiparasitic agent, an antifungal agent, an antiviral agent, an anti-neoplastic agent, an analgesic agent, an opioid, a drug for the treatment of arthritis, a drug for the treatment of rheumatoid arthritis, an antibody, a monoclonal antibody, a protein drug, a peptide drug, a gene, an enzyme, an antibiotic, a nucleic acid, a DNA, a RNA, a receptor, an antipsychotic, an anesthetic, a vaccine, a central nervous system agent, a growth factor, a hormone, an antihistamine, an osteoinductive agent, a cardiovascular agent, an anti-ulcer agent, a bronchodilator, a vasodilator, a birth control agent, a fertility enhancing agent, interferon alpha, a hormone, a growth hormone, an osteoporosis drug, parathyroid hormone, an obesity drug, a psychiatric drug, an anti-diabetes drug, a treatment for female infertility, an AIDS treatment, a hepatitis drug, a multiple sclerosis drug, a migraine headache drug, an allergic reaction treatment, interferon consensus, interleukin, erythropoietin, granulocyte-colony stimulating factor (GCSF), stem cell factor (SCI), leptin (OB protein), interferon (alpha, beta, gamma), ciprofloxacin, amoxycillin, lactobacillus, cefotaxime, levofloxacin, cefipime, mebendazole, ampicillin, lactobacillus, cloxacillin, norfloxacin, tinidazole, cefpodoxime, proxctil, azithromycin, gatifloxacin, roxithromycin, cephalosporin, anti-thrombogenics, aspirin, ticlopidine, sulfinpyrazone, heparin, warfarin, growth factors, differentiation factors, hepatocyte stimulating factor, plasmacytoma growth factor, brain derived neurotrophic factor (BDNF), glial derived neurotrophic factor (GDNF), neurotrophic factor 3 (NT3), fibroblast growth factor (FGF), transforming growth factor (TGF), platelet transforming growth factor, milk growth factor, endothelial growth factors (EGF), endothelial cell-derived growth factors (ECDGF), alpha-endothelial growth factors, beta-endothelial growth factor, neurotrophic growth factor, nerve growth factor (NGF), vascular endothelial growth factor (VEGF), 4-1 BB receptor (4-1BBR), TRAIL (TNF-related apoptosis inducing ligand), artemin (GFRalpha3-RET ligand), BCA-1 (B cell-attracting chemokine1), B lymphocyte chemoattractant (BLC), B cell maturation protein (BCMA), brain-derived neurotrophic factor (BDNF), bone growth factor such as osteoprotegerin (OPG), bone-derived growth factor, megakaryocyte derived growth factor (MGDF), keratinocyte growth factor (KGF), thrombopoietin, platelet-derived growth factor (PGDF), megakaryocyte derived growth factor (MGDF), keratinocyte growth factor (KGF), platelet-derived growth factor (PGDF), bone morphogenetic protein 2 (BMP2), BRAK, C-10, Cardiotrophin 1 (CT1), CCR8, anti-inflammatory: paracetamol, salsalate, diflunisal, mefenamic acid, diclofenac, piroxicam, ketoprofen, dipyrone, acetylsalicylic acid, antimicrobials amoxicillin, ampicillin, cephalosporins, erythromycin, tetracyclines, penicillins, trimethprim-sulfamethoxazole, quniolones, amoxicillin, clavulanatf, azithromycin, clarithromycin, anti-cancer drugs aliteretinoin, altertamine, anastrozole, azathioprine, bicalutamide, busulfan, capecitabine, carboplatin, cisplatin, cyclophosphamide, cytarabine, doxorubicin, epirubicin, etoposide, exemestane, vincristine, vinorelbine, hormones, thyroid stimulating hormone (TSH), sex hormone binding globulin (SHBG), prolactin, luteotropic hormone (LTH), lactogenic hormone, parathyroid hormone (PTH), melanin concentrating hormone (MCH), luteinizing hormone (LHb), growth hormone (HGH), follicle stimulating hormone (FSHb), haloperidol, indomethacin, doxorubicin, epirubicin, amphotericin B, Taxol, cyclophosphamide, cisplatin, methotrexate, pyrene, amphotericin B, anti-dyskinesia agents, Alzheimer vaccine, antiparkinson agents, ions, edetic acid, nutrients, glucocorticoids, heparin, anticoagulation agents, anti-virus agents, anti-HIV agents, polyamine, histamine and derivatives thereof, cystineamine and derivatives thereof, diphenhydramine and derivatives, orphenadrine and derivatives, muscarinic antagonist, phenoxybenzamine and derivatives thereof, protein A, streptavidin, amino acid, beta-galactosidase, methylene blue, protein kinases, beta-amyloid, lipopolysaccharides, eukaryotic initiation factor-4G, tumor necrosis factor (TNF), tumor necrosis factor-binding protein (TNF-bp), interleukin-1 (to 18) receptor antagonist (IL-Ira), granulocyte macrophage colony stimulating factor (GM-CSF), novel erythropoiesis stimulating protein (NESP), thrombopoietin, tissue plasminogen activator (TPA), urokinase, streptokinase, kallikrein, insulin, steroid, acetylsalicylic acid, acetaminophen, analgesic, anti-tumor preparation, anti-cancer preparation, anti-proliferative preparation or pro-apoptotic preparation.

In another embodiment, the active pharmaceutical ingredient of the drug delivery system described herein is a gonadotropin-releasing hormone (GnRH) agonist, deslorelin, naraferlin, leuprolide acetate, buserelin, a GnRH antagonist, azaline, acyline, degarelix, abarelix, cetrorelix, ganirelix, antide, a non-Peptide GnRH antagonist, a GnRH-toxin conjugate, a GnRH vaccine, an egg vaccine, a sperm vaccine, zona pellucida, a chemical sterilant, zinc solution, zinc gluconate, calcium chloride, chlorhexidine

digluconate, vinylcyclohexene diepoxide, hypertonic saline, an anti-androgen, an anti-estrogen, an aromatase inhibitor, a gene silencing agent, kisspeptin, a gonadotropin-inhibitory hormone, an egg protein, an egg peptide, a cytotoxin, a follicle-stimulating hormone receptor (FSHR) ligand-cytotoxin conjugate, or a retinoic acid receptor antagonist.

In yet another embodiment, the active pharmaceutical ingredient of the drug delivery system described herein is a gonadotropin-releasing hormone (GnRH) agonist, a GnRH antagonist, a non-Peptide GnRH antagonist, a GnRH-toxin conjugate, a GnRH vaccine, an egg vaccine, a sperm vaccine, a chemical sterilant, an anti-androgen, an anti-estrogen, an aromatase inhibitor, a gene silencing agent, kisspeptin, a gonadotropin-inhibitory hormone, an egg protein, an egg peptide, a cytotoxin, a follicle-stimulating hormone receptor (FSHR) ligand-cytotoxin conjugate, or a retinoic acid receptor antagonist.

In still another embodiment, the active pharmaceutical ingredient of the drug delivery system described herein is an anti-inflammatory agent, an antibacterial agent, an antiparasitic agent, an antifungal agent, an antiviral agent, an anti-neoplastic agent, an analgesic agent, an opioid, a drug for the treatment of arthritis, a drug for the treatment of rheumatoid arthritis, an antibody, a monoclonal antibody, a protein drug, a peptide drug, an antipsychotic, an anesthetic, a vaccine, a central nervous system agent, a growth factor, a hormone, an antihistamine, an osteoinductive agent, a cardiovascular agent, an anti-ulcer agent, a bronchodilator, a vasodilator, a birth control agent, a fertility enhancing agent, interferon alpha, a growth hormone, an osteoporosis drug, parathyroid hormone, an obesity drug, a psychiatric drug, an anti-diabetes drug, a treatment for female infertility, an AIDS treatment, a hepatitis drug, a multiple sclerosis drug, a migraine headache drug, or an allergic reaction treatment.

In yet another embodiment of the drug delivery system described herein, the solvent is selected from N-methyl-2-pyrrolidone (NMP), benzyl benzoate (BB), benzyl alcohol (BA), triethyl citrate (TEC), acetyl triethyl citrate (ATEC), and ethyl acetate (EA), or combinations thereof, the biodegradable polymer is selected from poly(L-lactic acid) and poly(D,L-lactic acid), or combinations thereof, and the active pharmaceutical ingredient is human progestogen, progesterone, norethisterone, ethynodiol diacetate, norethynodrel, dienogest, lynestrenol, medroxyprogesteroneacetate, megestroneacetate, levonorgestrel or levonorgestrel butanoate, norgestrel, desogestrel, gestodene, norgestimate, etonorgestrel, drospirenone, dienogest, or ethinylestradiol, or combinations thereof.

In still another embodiment, the active pharmaceutical ingredient of the drug delivery system described herein is a birth control agent.

In yet another embodiment, the birth control agent of the drug delivery system described herein is human progestogen, progesterone, norethisterone, ethynodiol diacetate, norethynodrel, dienogest, lynestrenol, medroxyprogesteroneacetate, megestroneacetate, levonorgestrel, levonorgestrel butanoate, norgestrel, desogestrel, gestodene, norgestimate, etonorgestrel, drospirenone, dienogest, ethinylestradiol, or combinations thereof.

In another embodiment, the birth control agent of the drug delivery system described herein is levonorgestrel or levonorgestrel butanoate.

In still another embodiment, the active pharmaceutical ingredient of the drug delivery system described herein is human progestogen, progesterone, norethisterone, ethynodiol diacetate, norethynodrel, dienogest, lynestrenol, medroxyprogesteroneacetate, megestroneacetate, levonorgestrel or levonorgestrel butanoate, norgestrel, desogestrel, gestodene, norgestimate, etonorgestrel, drospirenone, dienogest, or ethinylestradiol, or combinations thereof.

In yet another embodiment, the active pharmaceutical ingredient of the drug delivery system described herein is levonorgestrel or levonorgestrel butanoate.

In one embodiment, the active pharmaceutical ingredient of the drug delivery system described herein is a sterilant.

In an embodiment of the drug delivery system described herein, the system comprises poly(lactic-co-glycolic acid) (PLGA), poly(lactic acid) (PLA), levonorgestrel (LNG), N-methyl-2-pyrrolidone (NMP) and triethyl citrate (TEC).

In one embodiment of the drug delivery system described herein, the PLA comprises a first PLA having an inherent viscosity of about 0.40-0.70 dL/g and a second optional PLA having an inherent viscosity of about 0.40-0.70 dL/g that is different than the inherent viscosity of the first PLA. In another embodiment, the inherent viscosity of the first PLA is about 0.63 dL/g. In another embodiment, the inherent viscosity of the second PLA is about 0.47 dL/g.

In one embodiment of the drug delivery system described herein, the PLGA is comprised of approximately 50% lactic acid and approximately 50% glycolic acid.

In one embodiment of the drug delivery system described herein, the PLGA is comprised of approximately 50% lactic acid and approximately 50% glycolic acid.

In one embodiment of the drug delivery system described herein, the NMP and TEC are in a ratio of approximately 9:1, respectively.

In one embodiment of the drug delivery system described herein, the system is comprised of approximately 1-10% PLGA by weight, approximately 10-25% PLA by weight, 1-10% by weight LNG, and 55-88% NMP and TEC by weight.

In one embodiment of the drug delivery system described herein, the system is comprised of approximately 4% PLGA by weight, approximately 16-20% PLA by weight, 2.5-4% by weight LNG, and 72-77.5% NMP and TEC by weight.

In another embodiment of the drug delivery system described herein, the system comprises poly(lactic-co-glycolic acid) (PLGA), poly(lactic acid) (PLA), levonorgestrel (LNG), N-methyl-2-pyrrolidone (NMP) and ethyl acetate (EA).

In one embodiment of the drug delivery system described herein, the PLA comprises a first PLA having an inherent viscosity of about 0.40-0.70 dL/g and a second PLA having an inherent viscosity of about 0.40-0.70 dL/g that is different than the inherent viscosity of the first PLA. In another embodiment, the inherent viscosity of the first PLA is about 0.63 dL/g. In another embodiment, the inherent viscosity of the second PLA is about 0.47 dL/g.

In one embodiment of the drug delivery system described herein, the PLGA is comprised of approximately 50% lactic acid and approximately 50% glycolic acid.

In one embodiment of the drug delivery system described herein, the PLGA is comprised of approximately 50% lactic acid and approximately 50% glycolic acid.

In one embodiment of the drug delivery system described herein, the NMP and EA are in a ratio of approximately 9:1, respectively.

In one embodiment of the drug delivery system described herein, the system is comprised of approximately 1-10% PLGA by weight, approximately 10-25% PLA by weight, 1-10% by weight LNG, and 55-88% NMP and EA by weight.

In one embodiment of the drug delivery system described herein, the system is comprised of approximately 4% PLGA by weight, approximately 20% PLA by weight, 2.5-4% by weight LNG, and 72-73.5% NMP and EA by weight.

### Method of Treatment

Provided herein are methods of treatment related to administration of the injectable drug delivery system described herein.

In one embodiment, provided herein is a method of inducing amenorrhea, the method comprising administering to a subject in need thereof the injectable polymer matrix drug delivery system described herein.

In still another embodiment, provided herein is a method of reducing or inhibiting spermatogenesis, the method comprising administering to a subject in need thereof the injectable polymer matrix drug delivery system described herein.

In yet another embodiment, provided herein is a method of minimizing uterine bleeding, the method comprising administering to a subject in need thereof the injectable polymer matrix drug delivery system described herein.

In another embodiment, provided herein is a method of minimizing estrus, the method comprising administering to a subject in need thereof the injectable polymer matrix drug delivery system described herein.

In another embodiment, provided herein is a method of administering the drug delivery system described herein, wherein upon administration to the subject in need thereof, the active pharmaceutical ingredient is continuously released at a rate according to a zero order reaction from about 0 months to about 18 months.

In another embodiment, provided herein is a method of administering the drug delivery system described herein, wherein upon administration to the subject in need thereof, the active pharmaceutical ingredient is released for at least 3 months.

The term “subject” refers to a human subject or a non-human subject. In some embodiments, the subject is human. When the subject is a non-human subject, non-limiting examples include another mammalian species or an avian species. Examples of mammalian subjects include a mouse, a rabbit, a rat, a transgenic non-human animal, a domestic animal such as a dog or a cat, or farmed animals such as cows, horses, pigs, sheep, goats.

In another embodiment, provided herein is a method of administering the drug delivery system described herein, wherein the polymer matrix is injected through a needle of about 18-gauge to about 26-gauge.

In another embodiment, provided herein is a method of administering the drug delivery system described herein, wherein the polymer matrix is injected through a needle of about 21-gauge.

In another embodiment, provided herein is a method of administering the drug delivery system described herein, wherein the polymer matrix is injected through a needle of about 22-gauge.

In another embodiment, provided herein is a method of administering the drug delivery system described herein, wherein the polymer matrix is injected through a needle of about 23-gauge to about 26-gauge.

In another embodiment, provided herein is a method of administering the drug delivery system described herein, wherein the polymer matrix is injected through a needle of about 23-gauge.

In another embodiment, provided herein is a method of administering the drug delivery system described herein, wherein the system is formulated for subcutaneous injection or intramuscular injection.

In another embodiment, provided herein is a method of administering the drug delivery system described herein, wherein the system forms a semi-solid or solid depot at the injection site.

In another embodiment, provided herein is a method of forming a polymer matrix drug delivery system described herein comprising:

a) adding an active pharmaceutical ingredient to a solvent or a combination of solvents;

b) dissolving or dispersing the active pharmaceutical ingredient;

c) adding the dissolved or dispersed active pharmaceutical solution to a biodegradable polymer selected from the group consisting of poly(lactic-co-glycolic acid), poly(lactic acid), poly(ε-caprolactone), poly(ethylene glycol-block-lactic acid), poly(alkylcyanoacrylate), polyanhydride, poly(bis(p-carboxyphenoxy) propane-sebacic acid), polyorthoester, polyphosphoester, polyphosphazene, polyurethane, and poly(amino acid), or combinations thereof; and

d) mixing the dissolved or dispersed active pharmaceutical ingredient and biodegradable polymer solution to homogeneity;

such that the polymer matrix drug delivery system is formed.

In another embodiment, provided herein is a method of administering the drug delivery system described herein, wherein the drug delivery system comprises a formulation selected from any one of tables 1, 1b, 1c, 2, 4, 5, 6 and 8.

In one embodiment, provided herein is a method of inducing amenorrhea, the method comprising administering to a subject in need thereof the injectable polymer matrix drug delivery system comprising poly(lactic-co-glycolic acid) (PLGA), poly(lactic acid) (PLA), levonorgestrel (LNG), N-methyl-2-pyrrolidone (NMP) and triethyl citrate (TEC).

In one embodiment of the methods described herein, the PLA comprises a first PLA having an inherent viscosity of about 0.40-0.70 dL/g and a second optional PLA having an inherent viscosity of about 0.40-0.70 dL/g that is different than the inherent viscosity of the first PLA. In another embodiment, the inherent viscosity of the first PLA is about 0.63 dL/g. In another embodiment, the inherent viscosity of the second PLA is about 0.47 dL/g.

In one embodiment of the methods described herein, the PLGA is comprised of approximately 50% lactic acid and approximately 50% glycolic acid.

In one embodiment of the methods described herein, the PLGA is comprised of approximately 50% lactic acid and approximately 50% glycolic acid.

In one embodiment of the methods described herein, the NMP and TEC are in a ratio of approximately 9:1, respectively.

In one embodiment of the methods described herein, the system is comprised of approximately 1-10% PLGA by weight, approximately 10-25% PLA by weight, 1-10% by weight LNG, and 55-88% NMP and TEC by weight.

In one embodiment of the methods described herein, the system is comprised of approximately 4% PLGA by weight, approximately 16-20% PLA by weight, 2.5-4% by weight LNG, and 72-77.5% NMP and TEC by weight.

In another embodiment, provided herein is a method of inducing amenorrhea, the method comprising administering to a subject in need thereof the injectable polymer matrix drug delivery system comprising poly(lactic-co-glycolic acid) (PLGA), poly(lactic acid) (PLA), levonorgestrel (LNG), N-methyl-2-pyrrolidone (NMP) and ethyl acetate (EA).

In one embodiment of the methods described herein, the PLA comprises a first PLA having an inherent viscosity of about 0.40-0.70 dL/g and a second PLA having an inherent viscosity of about 0.40-0.70 dL/g that is different than the inherent viscosity of the first PLA. In another embodiment, the inherent viscosity of the first PLA is about 0.63 dL/g. In another embodiment, the inherent viscosity of the second PLA is about 0.47 dL/g.

In one embodiment of the methods described herein, the PLGA is comprised of approximately 50% lactic acid and approximately 50% glycolic acid.

In one embodiment of the methods described herein, the NMP and EA are in a ratio of approximately 9:1, respectively.

In one embodiment of the methods described herein, the drug delivery system is comprised of approximately 1-10% PLGA by weight, approximately 10-25% PLA by weight, 1-10% by weight LNG, and 55-88% NMP and EA by weight.

In one embodiment of the methods described herein, the drug delivery system is comprised of approximately 4% PLGA by weight, approximately 20% PLA by weight, 2.5-4% by weight LNG, and 72-73.5% NMP and EA by weight.

### EXEMPLIFICATION

### Example 1

### Injectability

Based on the tamarin (Prototype A) and rat (Protype B) formulations, we used polyesters with lower viscosity, adjusted the ratios between the polyesters, used different combination of solvents N-methyl-2-pyrrolidone (NMP) and/or triethyl citrate (TEC), acetyl triethyl citrate (ATEC), benzyl benzoate (BB), and benzyl alcohol (BA), and were able to develop 5 formulations that could be injected through 22 gauge needles (highlighted in bold Table 1A).

TABLE 1A
Injectability of Polymer Solutions.

| Polymer | Injectability | |
|---|---|---|
| Solution # | Gauge 22 | Composition |
| 1 | Hard +++ | 32 wt % PLGA50 (0.65)/64 wt % NMP (Prototype B: rat |
| | | formulation without drug) |
| 4 | Easy + | 4.7 wt % PLGA 85 (0.65) + 18.8 wt % PLA (0.25) + 70.5 wt % |
| | | NMP/TEC (9/1 w/w). |
| 6 | Easy ++ | 23.5 wt % PLA (0.25) + 70.5 wt % NMP/TEC (9/1 w/w) |
| 16 | Hard + | 23.5 wt % PLGA85 (0.65) + 70.5 wt % BB |
| 17 | Hard + | 23.5 wt % PLGA85 (0.65) + 70.5 wt % BA |
| 18 | — | 40 wt % PLA (0.25) + 60 wt % ATEC |
| 19 | — | 40 wt % PLA (0.25) + 60 wt % BB |
| 20 | Hard + | 40 wt % PLA (0.25) + 60 wt % BA |
| 24 | Hard + | 40 wt % PLA (0.25) + 60 wt % NMP/ATEC (9/1 w/w) |
| 25 | Hard + | 40 wt % PLA (0.25) + 60 wt % NMP/BB (9/1 w/w) |
| 26 | Hard 0 | 40 wt % PLA (0.25) + 60 wt % NMP/BA (9/1 w/w) |
| 27 | Easy + | 11.75 wt % PLGA85(0.65) + 11.75 wt % PLA (0.25) + 70.5 wt % |
| | | NMP/ATEC (9/1 w/w) |
| 28 | Easy ++ | 11.75 wt % PLGA85 (0.65) + 11.75 wt % PLA (0.25) + 70.5 wt % |
| | | NMP/BB (9/1 w/w) |
| 29 | Easy ++ | 11.75 wt % PLGA85 (0.65) + 11.75 wt % PLA (0.25) + 70.5 wt % |
| | | NMP/BA (9/1 w/w) |
| 38 | Hard + | 4.7 wt % PLGA 50 (0.65) + 18.8 wt % PLA (0.65) + 70.5 wt % |
| | | NMP/TEC (9/1 w/w) (Prototype A: tamarin formulation |
| | | without drug) |

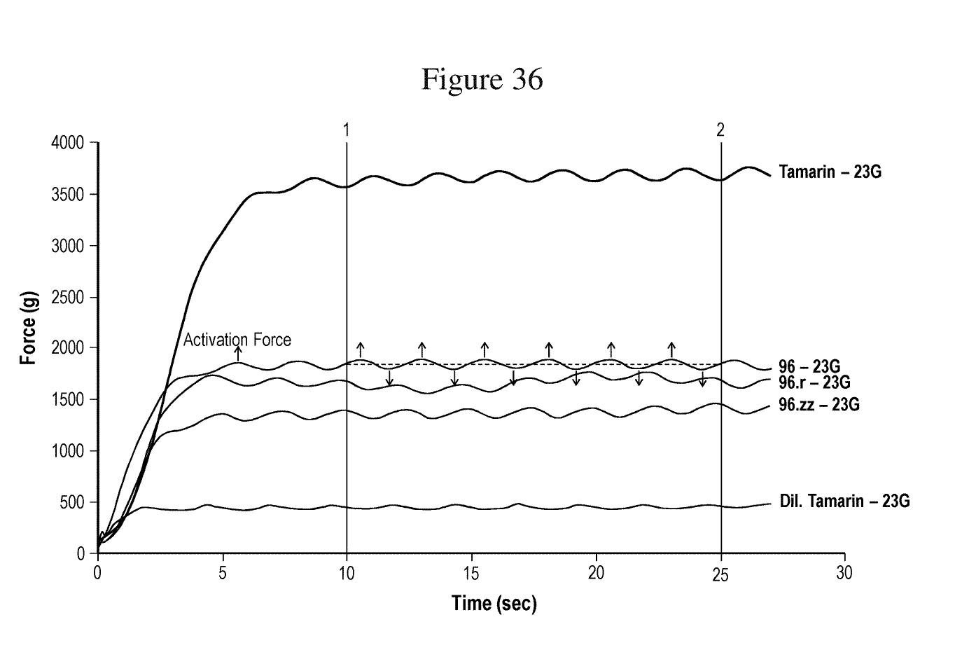

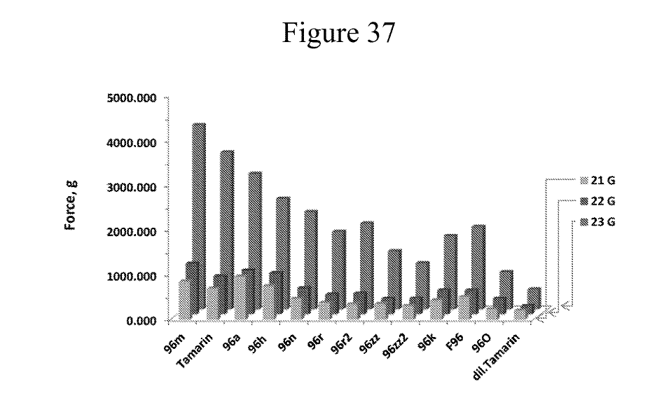

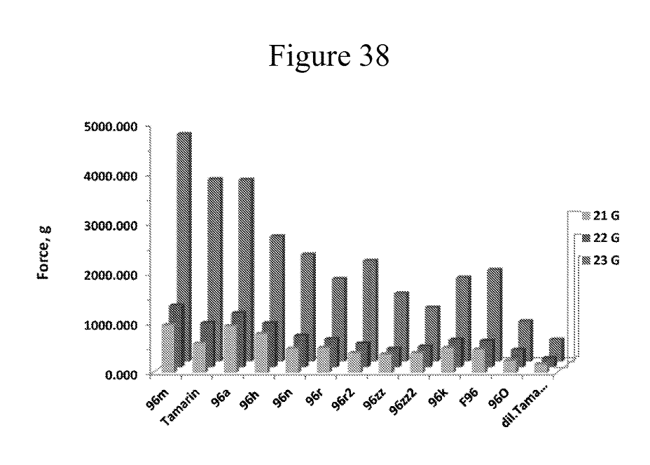

The formulations listed in Table 1B were tested for their injectability through 23, 22 and 21G needles by measuring injection force using a TA texture analyzer. Around 0.5 mL of formulation was taken into 1 mL syringe and was positioned in the holder with downward needle. 5-kg loading cell was placed in contact with the plunger end of the syringe and test was carried out at a crosshead speed of 1 mm·s−1, representative of manual syringe delivery to patient. The force required to displace the plunger was measured as a function of plunger displacement (mm) (FIGS. 36-38). The following two parameters were estimated from the plot: activation force—the initial force required to move the plunger and average force or gliding force—the force required to sustain the movement in the plunger at required crosshead speed. The results show that the injection force data correlated well with the shear viscosity data: the higher is the viscosity, the higher is the injection force. The injection forces required for the formulations to go through the 22G and 21G needles were similar and about three times lower than with the 23G needles.

TABLE 1B
Activation force and average force required to expel liquid for different formulations.

| | Shear | 23G | | 22G | | 21G | |
|---|---|---|---|---|---|---|---|
| | Viscosity | Activation | Average | Activation | Average | Activation | Average |
| Test ID | Pa · s | Force (g) | Force (g) | Force (g) | Force (g) | Force (g) | Force (g) |
| NMP TEC | | 78.517 ± | 91.302 ± | | | | |
| | | 1028 | 6.58 | | | | |
| NMP EA | | 135.170 ± | 136.26 ± | | | | |
| | | 5.91 | 4.14 | | | | |
| NMP BB | | 115.796 ± | 88.53 ± | | | | |
| | | 15.04 | 4.72 | | | | |
| 96.m | 2.14 | 4124.39 ± | 4568.67± | 1134.72 ± | 1241.77 ± | 851.18 ± | 963.64 ± |
| | | 20.6 | | 84.37 | 85.32 | 50.47 | 41.27 |
| Tamarin | 1.7 | 3510.66 ± | 3669.53± | 859.16 ± | 891.51 ± | 689.89 ± | 591.88 ± |
| | | 205.15 | | 226.48 | 38.26 | 94.27 | 65.08 |
| 96.a | 1.5 | 3035.66 ± | 3655.08 ± | 991.03 ± | 1088.98 ± | 961.35 ± | 943.69 ± |
| | | 470.49 | 1164.18 | 67.19 | 98.65 | 61.52 | 32.74 |
| 96.h | 1.09 | 2477.95 ± | 2518.53± | 927.04 ± | 889.97 ± | 752.85 ± | 785.65 ± |
| | | 182.74 | | 64.85 | 60.68 | 34.81 | 33.91 |
| 96.n | 0.97 | 2180.55 ± | 2154.09± | 594.62 ± | 633.14 ± | 466.59 ± | 491.36 ± |
| | | 84.72 | | 67.35 | 65.91 | 29.84 | 51.08 |
| 96.r | 0.75 | 1736.71 ± | 1665.03± | 446.93 ± | 568.76 ± | 376.99 ± | 505.32 ± |
| | | 90.67 | | 90.18 | 68.53 | 57.5 | 69.43 |
| 96.r2 | 0.76 | 1922.31 ± | 2030.68± | 473.64 ± | 482.53 ± | 341.314 ± | 395.54 ± |
| | | 60.37 | | 43.18 | 73.85 | 39.72 | 47.28 |
| 96.zz | 0.66 | 1304.34 ± | 1373.64 ± | 352.76 ± | 378.71 ± | 352.59 ± | 373.24 ± |
| | | 80.18 | 81.62 | 36.59 | 58.67 | 25.46 | 30.84 |
| 96.zz2 | 0.63 | 1041.33 ± | 1092.73 ± | 361.44 ± | 419.66 ± | 310.28 ± | 396.50 ± |
| | | 42.94 | 68.76 | 41.63 | 49.83 | 74.38 | 16.85 |
| 96.k | 0.64 | 1649.34 ± | 1690.67± | 539.11 ± | 554.95 ± | 438.22 ± | 497.76 ± |
| | | 50.64 | | 25.68 | 50.94 | 61.34 | 34.34 |
| 96 | 0.67 | 1850.29 ± | 1842.39 ± | 542.83 ± | 535.94 ± | 500.00 ± | 473.88 ± |
| | | 14.64 | 92.31 | 45.82 | 30.18 | 28.36 | 46.13 |
| 96.O | 0.33 | 837.69 ± | 812.60 ± | 356.85 ± | 349.56 ± | 237.71 ± | 247.36 ± |
| | | 15.07 | 85.29 | 30.12 | 40.27 | 62.47 | 38.17 |
| dil.Tamarin | 0.12 | 448.41 ± | 447.95 ± | 193.65 ± | 188.275 ± | 204.72 ± | 177.39 ± |
| | | 45.18 | 89.96 | 18.27 | 12.59 | 18.61 | 42.68 |

Formulations listed in Table 1C were prepared based on the 96 formulation by altering the polymer and drug contents and polymer intrinsic viscosity. The injectability of the new formulations through four size gauge needles: 18, 21, 22 and 23 G was tested and the results were listed in Table 1D. Based on previous in vitro accelerated release method development results, a release medium containing PBS (pH 9), 25% ethanol and 0.5% tween 20 was selected for conducting in vitro accelerated release study of the new formulations for initial burst study. Release samples were collected at release time points, 10, 20 and 30 min and 1, 1.5, 2, 2.5, 3, 4, 5, 6 and 24 h for analysis by HPLC (results not shown).

TABLE 1C
Formulations tested for accelerated release studies.

| | PLGA 50:50 | PLA | PLA | | Solvents |
|---|---|---|---|---|---|
| Sample | (IV 0.63) | (IV 0.47) | (IV 0.63) | LNG | (9:1 w:w) |
| ID | wt % | wt % | wt % | wt % | wt % |
| 96.a | 4 | | 20 | 2.5 | 73.5 |
| | | | | | NMP/BB |
| 96.b | 4 | | 16 | 2.5 | 77.5% |
| | | | | | NMP/BB |
| 96.c | 4 | | 12 | 2.5 | 81.5% |
| | | | | | NMP/BB |
| 96.j | 4 | | 20 | 2.5 | 77.5% |
| | | | | | NMP/TEC |
| 96.k | 4 | | 16 | 2.5 | 77.5% |
| | | | | | NMP/TEC |
| 96.l | 4 | | 12 | 2.5 | 81.5% |
| | | | | | NMP/TEC |

TABLE 1D
Injectability of formulations through different gauge needles.

| Sample ID | 18 G | 21 G | 22G | 23 G |
|---|---|---|---|---|
| 96.a | 5 | 4 | 4 | 3 |
| 96.b | 5 | 4 | 4 | 3 |
| 96.c | 5 | 5 | 5 | 4 |
| 96.j | 5 | 4 | 4 | 3 |
| 96.k | 5 | 4 | 4 | 3 |
| 96.l | 5 | 5 | 5 | 4 |

The injectability was rated on a scale from 0-5 (0-Harder; 5-Easier).

### Example 2

### Viscosity

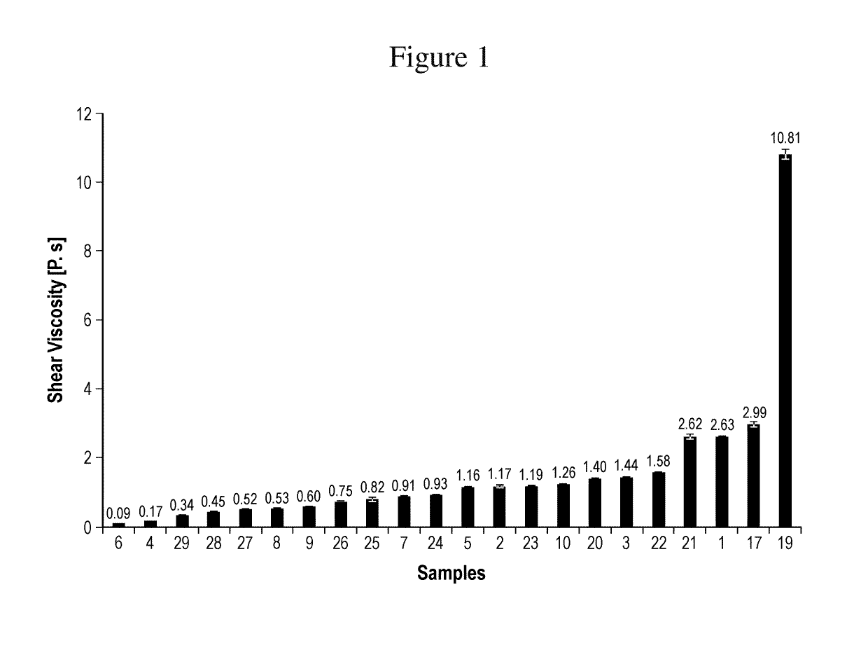

All the viscosity measurements of different polymer solutions were tabulated (Table 2). Most of the solutions exhibited Newtonian flow (viscosity remained constant over the range of shear rate) after the yield stress was applied. Sample 17 and 19 showed a gradual decrease in viscosity with increasing shear rate. Solutions 16, 18 and 30 were exceptions; these solutions showed decreased viscosity with increase in the shear rate. FIG. 1 displays the viscosity of polymer solutions.

TABLE 2
Viscosity of Polymer Solutions.

| | | | | PLA | PLA | | |
|---|---|---|---|---|---|---|---|
| | | PLGA | PLGA | 100 | 100 | Viscosity | |
| Sample | Solvent | 50:50:00 | 85:15:00 | (0.25) | (0.65) | [Pa · s] | STD |

| | | | | | | |
|---|---|---|---|---|---|---|
| #1 | 67 wt % NMP | 33 | wt % | | 2.63 | 0.02 |
| #2 | 70.5 wt % 10% TEC/90% NMP | 4.7 | wt % | 18.8 | 1.17 | 0.03 |

| #3 | 70.5 wt % 10% TEC/90% NMP | 4.7 | wt % | | | 18.8 | 1.44 | 0.01 |
|---|---|---|---|---|---|---|---|---|
| #4 | 70.5 wt % 10% TEC/90% NMP | 4.7 | wt % | 18.8 | wt % | | 0.17 | 0.00 |
| #5 | 70.5 wt % 10% TEC/90% NMP | 23.5 | wt % | | | | 1.16 | 0.02 |
| #6 | 70.5 wt % 10% TEC/90% NMP | | | 23.5 | wt % | | 0.09 | 0.00 |
| #7 | 60 wt % 10% TEC/90% NMP | | | 40 | wt % | | 0.91 | 0.01 |
| #8 | 70.5 wt % 10% TEC/90% NMP | 11.75 | | 11.75 | wt % | | 0.53 | 0.01 |
| #9 | 70.5 wt % 10% TEC/90% NMP | 11.75 | | 11.75 | wt % | | 0.60 | 0.00 |
| #10 | 65.8 wt % 10% TEC/90% NMP | 14.1 | | 14.1 | wt % | | 1.26 | 0.01 |
| #16 | 70.5 wt % benzyl benzoate | 23.5 | wt % | | | | | |
| #17 | 70.5 wt % benzyl alcohol | 23.5 | wt % | | | | 2.992 | 0.048 |
| #18 | 60 wt % acetyl triethyl citrate | | | 40 | wt % | | | |
| #19 | 60 wt % benzyl benzoate | | | 40 | wt % | | 10.81 | 0.16 |
| #20 | 60 wt % benzyl alcohol | | | 40 | wt % | | 1.40 | 0.01 |
| #21 | 70.5 wt % NMP(90)/acetyl | 23.5 | wt % | | | | 2.62 | 0.06 |
| | triethyl citrate (10) | | | | | | | |
| #22 | 70.5 wt % NMP (90)/benzyl | 23.5 | wt % | | | | 1.58 | 0.01 |
| | benzoate (10) | | | | | | | |
| #23 | 70.5 wt % NMP(90)/benzyl | 23.5 | wt % | | | | 1.19 | 0.00 |
| | alcohol (10) | | | | | | | |
| #24 | 60 wt % NMP(90)/acetyl | | | 40 | wt % | | 0.93 | 0.03 |
| | triethyl citrate (10) | | | | | | | |
| #25 | 60 wt % NMP (90)/benzyl | | | 40 | wt % | | 0.82 | 0.02 |
| | benzoate (10) | | | | | | | |
| #26 | 60 wt % NMP(90)/benzyl | | | 40 | wt % | | 0.75 | 0.01 |
| | alcohol (10) | | | | | | | |
| #27 | 70.5 wt % NMP(90)/acetyl | 11.75 | wt % | 11.75 | wt % | | 0.52 | 0.01 |
| | triethyl citrate (10) | | | | | | | |
| #28 | 70.5 wt % NMP (90)/benzyl | 11.75 | wt % | 11.75 | wt % | | 0.45 | 0.01 |
| | benzoate (10) | | | | | | | |
| #29 | 70.5 wt % NMP(90)/benzyl | 11.75 | wt % | 11.75 | wt % | | 0.34 | 0.00 |
| | alcohol (10) | | | | | | | |

| | | |
|---|---|---|
| #30 | 70.5 wt % acetyl TEC | 23.5 |

### Example 3

### Solubility

Solubility of LNG and LNG-B in single solvents and co-solvents at both 21 and 37° C. is listed in the below table (Table 3). The solubility of LNG-B is lower than LNG in the solvents except for BB and TEC. The solubility of LNG and LNG-B at 21° C. is lower than that at 37° C. in general.

TABLE 3
Solubility of LNG and LNG-B in Different Solvents at 21 and 37° C.

| | LNG solubility, mg/ml | | LNG-B solubility, mg/ml | |
|---|---|---|---|---|
| Solvent | 21° C. | 37° C. | 21° C. | 37° C. |
| NMP | 126.7 ± 0.3 | 133.61 ± 0.39 | 37.6 ± 0.4 | 41.8 ± 0.1 |
| BA | N/A | 47.65 ± 0.15 | N/A | N/A |
| BB | 6.3 ± 0.1 | 8.25 ± 0.02 | 17.3 ± 0.1 | 21.8 ± 0.1 |
| TEC | 2.5 ± 0.2 | 3.97 ± 0.02 | 3.4 ± 0.2 | 4.4 ± 0.1 |
| ATEC | ~2* | ~2* | </~4* | </~4* |
| NMP/ | 118.9 ± 0.1 | 135.6 ± 0.1 | 21.9 ± 0.2 | 33.5 ± 0.1 |
| BB | | | | |
| (9:1) | | | | |
| NMP/ | 101.7 ± 0.1 | 116.6 ± 0.1 | 21.5 ± 0.1 | 33.2 ± 0.1 |
| TEC | | | | |
| (9:1) | | | | |
| NMP/ | N/A | 128.0 ± 1.4 | 58.2 ± 0.1** | 65.5 ± 0.1** |
| ATEC | | | | |
| (9:1) | | | | |

*This value is based on observation since no calibration curve was successfully built for the particular system.
**This value is based on the reading.

### Example 4

### In Vitro Release

Surprisingly, sustained release of the active pharmaceutical ingredient of the drug delivery system is observed. Critically, the active pharmaceutical is observed to be released at an approximately constant rate in addition to a sustained manner. Thus, dosage may be properly determined based on a given subjects physical characteristics. Sustained and steady release of a drug for at least up to six months is preferred, particularly when developing a contraceptive.

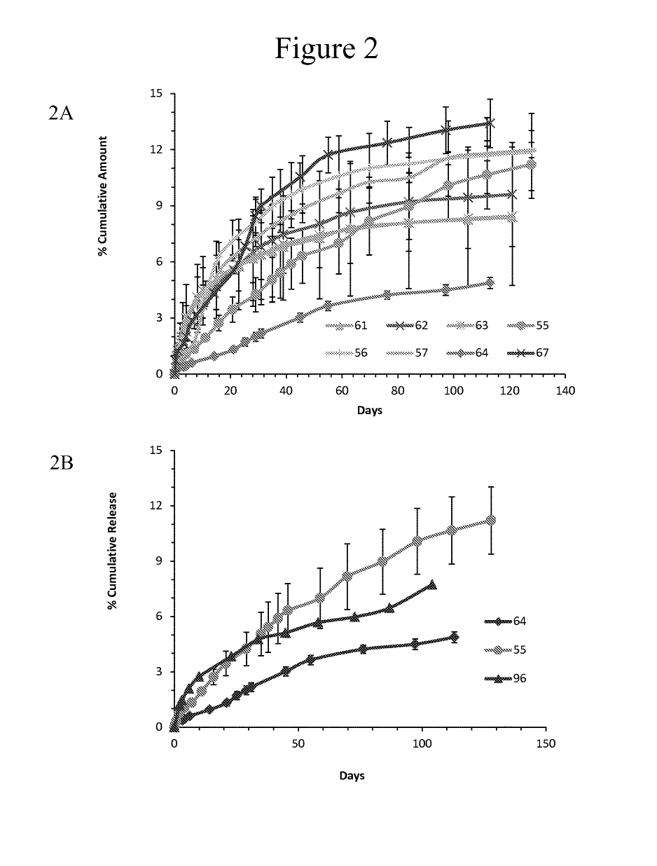

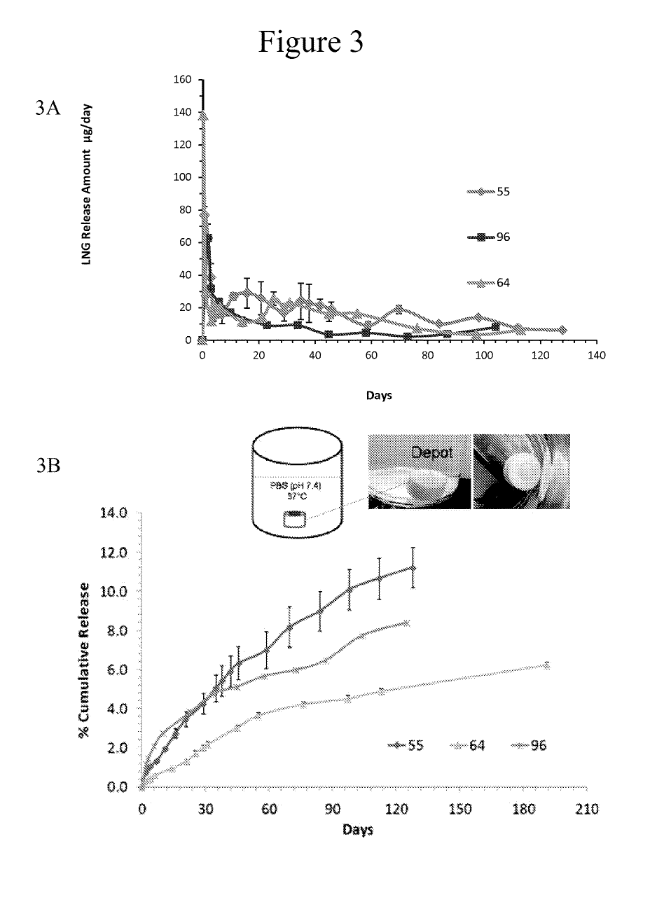

Nine formulations (Table 4A) have been studied for in intro release. FIGS. 2A, 2B, 3A, and 3B show that LNG is continuously released from each of the nine formulations for 3.5 to 4.5 months. Among the nine formulations, 64 (tamarin) shows the slowest LNG release. The formulation 55 continuously shows near zero order LNG release at about 10 ug LNG per day.

TABLE 4A
Nine Formulations for In Vitro Release Study.

| | | | PLA | | | |
|---|---|---|---|---|---|---|
| | PLGA 50:50 | PLGA 85:15 | (0.25) | PLA (0.63) | LNG | Solvent (9:1 |
| Formulation# | (0.63) wt % | (0.63) wt % | wt % | wt % | wt % | w:w) wt % |
| 55 | | | 12% | 12% | 4% | 72% NMP/BB |
| 56 | | | 12% | 12% | 4% | 72% NMP/BA |
| 57 | | | 12% | 12% | 4% | 72% |
| 61 | | | 12% | 12% | 4% | 72% NMP/EA |
| 62 | | | 12% | 12% | 4% | 72% NMP/TEC |
| 63 | | 12% | | 12% | 4% | 72% NMP/EA |
| 64 | 4.7% | | | 18.8% | 6% | 70.5% |
| 67 | | 12.2% | 12.2% | | 2.5% | 73.1% |
| 68 | | 12.2% | 12.2% | | 2.5% | 73.1% NMP/BB |

Abbreviations:
N-methyl-2-pyrrolidone (NMP), BB—benzyl benzoate, BA—benzyl alcohol, TEC—triethyl citrate, ATEC—acetyl triethyl citrate, EA—ethyl acetate.

FIGS. 2A and 2B show in vitro cumulative release of LNG from formulations 55, 56, 57, 61, 62, 63, 64, 67 and 96. FIGS. 3A and 3B show in vitro daily release of LNG from formulations 55, 64 (tamarin) and 96. Data from formulations 55, 64 (tamarin) and 96 were fit to the Korsmeyer-Peppas model (equation 1), which describes drug release from a polymeric system. Formula 55 and formula 96 released LNG in vitro for 3-4 months without losing integrity, and formula 64 released LNG for more than 6 months without losing integrity (FIG. 3B).

Mₜ/M_{∞}=Ktⁿ (1)

TABLE 4B

| | |
|---|---|
| Formulation | Korsmeyer-Peppas (eqn. 1) |

| # | R² | n |
|---|---|---|
| 55 | 0.995 | 0.693 |
| 64 | 0.969 | 0.634 |
| 96 | 0.995 | 0.456 |

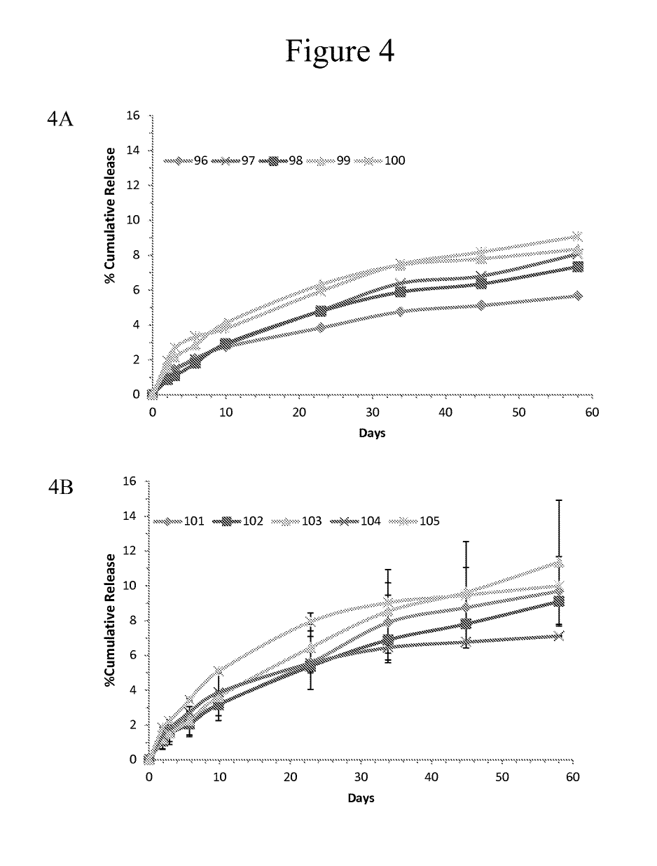

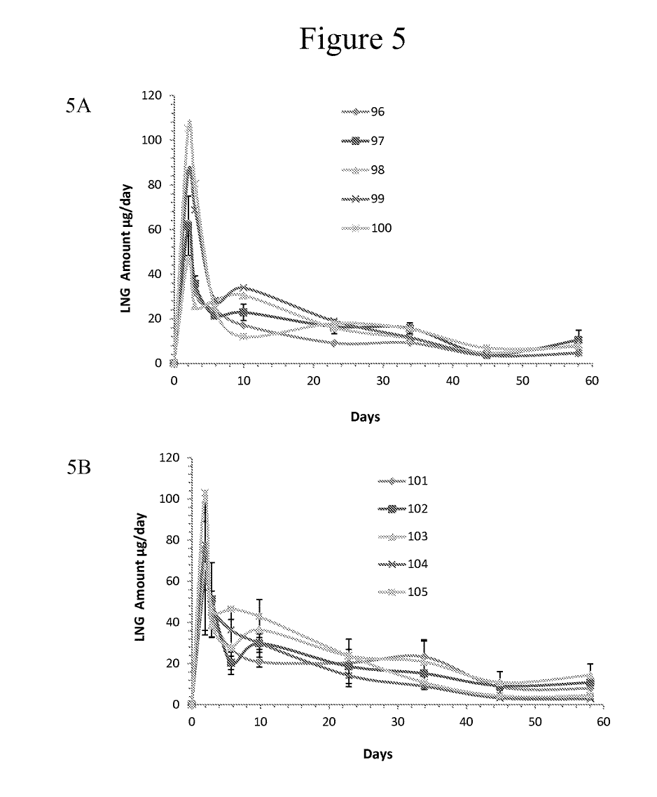

Ten formulations listed in Table 5 have also been studied for in intro release. FIGS. 4A, 4B, 5A, and 5B show that LNG is continuously slowly released from each of the night formulations with above 4 μg per day for two months. When the BB content in the mixture solvent of NMP and BB is increased from 10% to 30% (96-97, and 101-103), the initial burst release is decreased. The formulations containing PLGA50 (96-100) have slower release of LNG than the corresponding ones containing PLGA85. The formulation containing NMP/BB (96-98, and 101-103) show slightly more steady release per day after the initial burst than the other formulations containing TEC or ATEC as a solvent.

TABLE 5
Second Batch Formulations for In Vitro Release Study.

| | PLA | PLGA | NMP/BB | NMP/BB | NMP/BB | NMP/TEC | NMP/ATEC | |
|---|---|---|---|---|---|---|---|---|
| Formulation # | (0.47) | (0.63 | (9:1) | (8:2) | (7:3) | (9:1) | (9:1) | LNG |
| 96 | 20.3% | 4.1% | 73.1% | | | | | 2.5% |
| 97 | 20.3% | 4.1% | | 73.1% | | | | 2.5% |
| 98 | 20.3% | 4.1% | | | 73.1% | | | 2.5% |
| 99 | 20.3% | 4.1% | | | | 73.1% | | 2.5% |
| 100 | 20.3% | 4.1% | | | | | 73.1% | 2.5% |
| 101 | 20.3% | 4.1% | 73.1% | | | | | 2.5% |
| 102 | 20.3% | 4.1% | | 73.1% | | | | 2.5% |
| 103 | 20.3% | 4.1% | | | 73.1% | | | 2.5% |
| 104 | 20.3% | 4.1% | | | | 73.1% | | 2.5% |
| 105 | 20.3% | 4.1% | | | | | 73.1% | 2.5% |

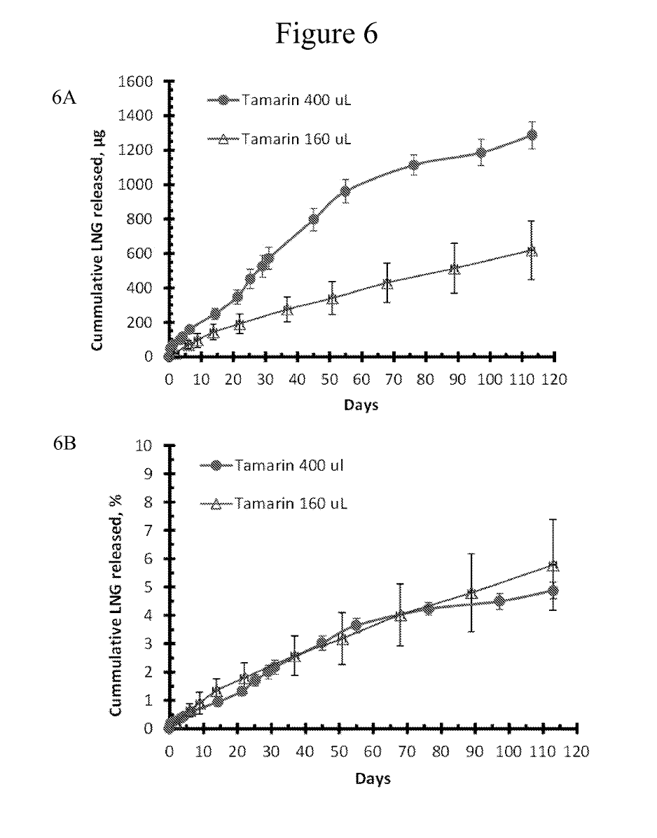

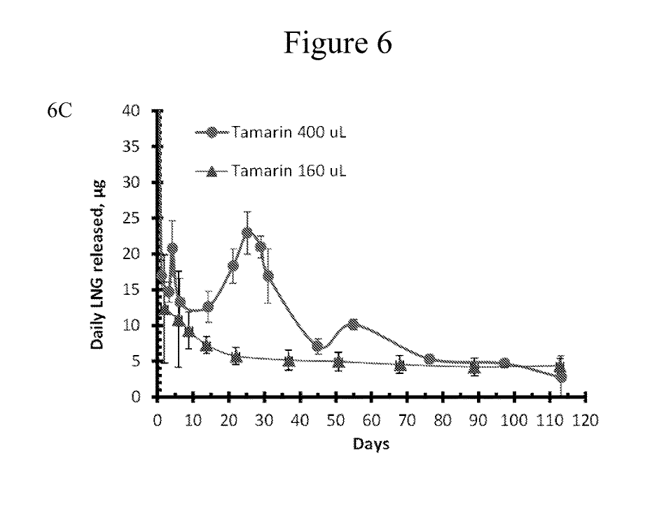

FIGS. 4A and 4B show in vitro LNG release amount per day from formulations 96-105. FIGS. 5A and 5B show in vitro LNG release amount per day from formulations 96-105. The LNG release profiles from the tamarin formulation in two different volumes 160 and 400 μL have been generated in FIGS. 6A, 6B, and 6C. The results show that the 400 μL formulation released more LNG than the 160 μL one, but that both formulations released LNG at about the same rate.

### Example 5

### In Vivo LNG Plasma Concentration

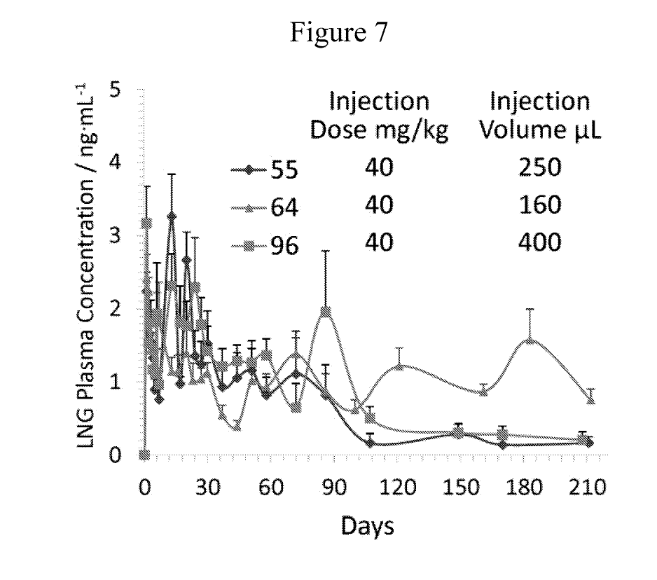

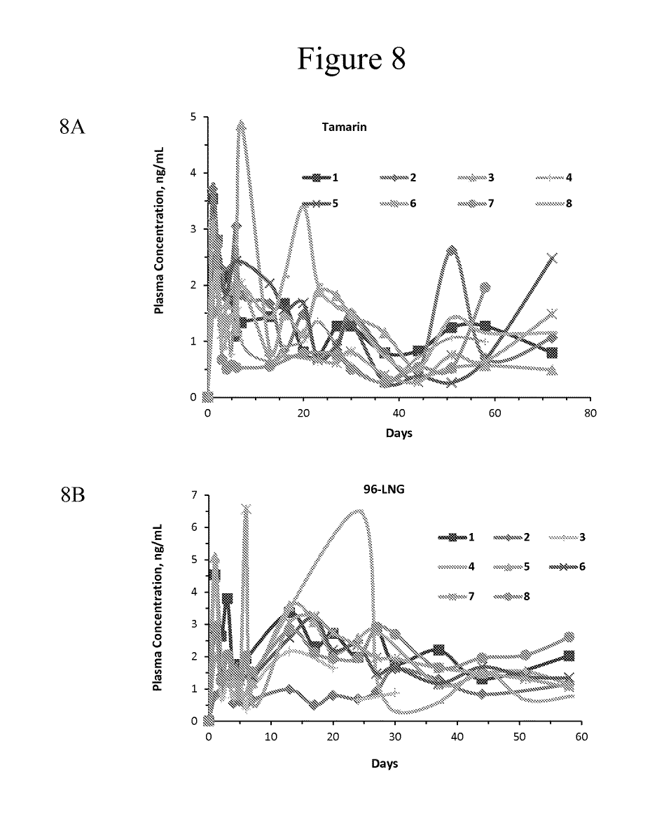

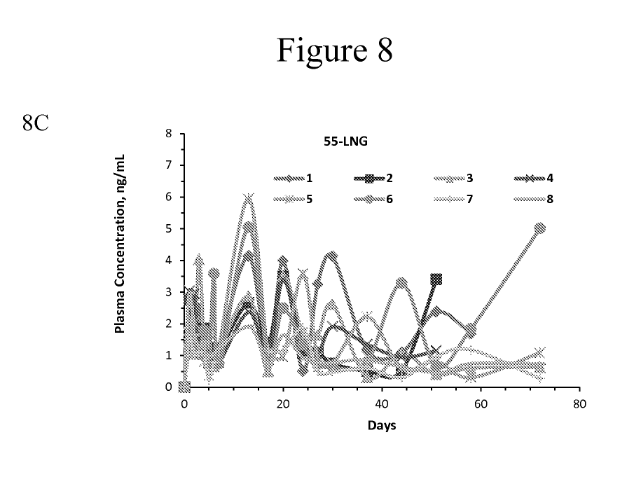

Tamarin-LNG (64), 55-LNG, 96-LNG and 55-LNG-B formulations were injected subcutaneously into female rats at 8 rats per formulation and roughly 40 mg/kg dose. Blood was collected after the injection as a function of time. Plasma was isolated from all the blood samples and stored at −80° C. for PK studies using UFLC/MS/MS. FIG. 7 shows that 0.2-4 ng/mL LNG plasma concentration was detected for seven months after sub-Q injection of tamarin (64), 55 LNG and 96 LNG formulations into the rats at 10 mg/rat (roughly 40 mg/kg). FIGS. 8A, 8B, and 8C show individual rat plasma concentrations in all 3 formulations (tamarin (64), 96, and 55). Each point represents the mean±SE, n=8.

### Example 6

### Animal Body Weight

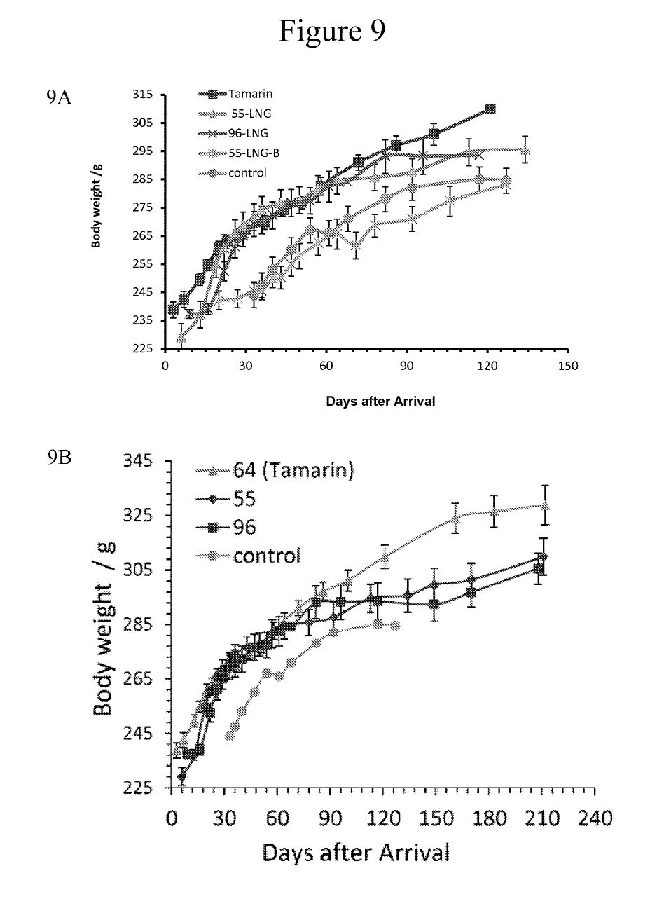

The body weights of the rats subcutaneously injected with tamarin, 55 LNG, 96 LNG and 55 LNG-B formulations have been measured at the same time intervals when the blood samples are collected. FIGS. 9A and 9B show that all the rats gain weight over time. The rats injected with tamarin, 55 LNG and 96 LNG formulations gained similar weight among themselves but more weight than the control rats at most time points. However, the rats injected with 55-LNG-B formulation gained comparable or slightly lower weight than the control rats. The reasons for the above body weight changes are not very clear at this moment.

### Example 7

### Vaginal Cytology

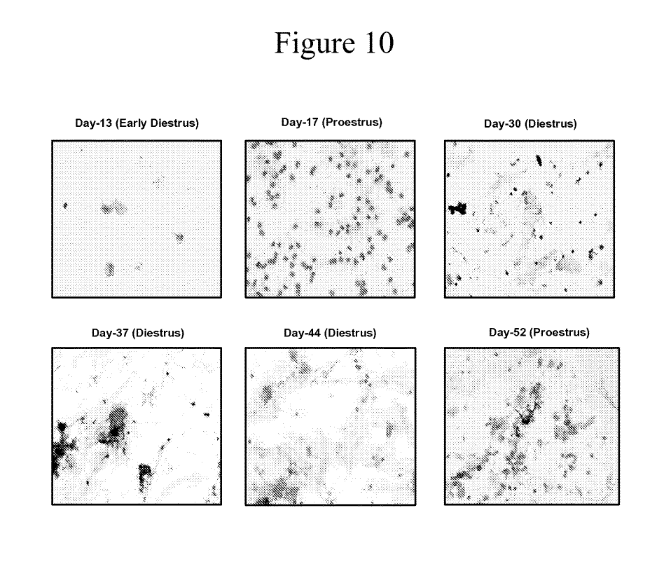

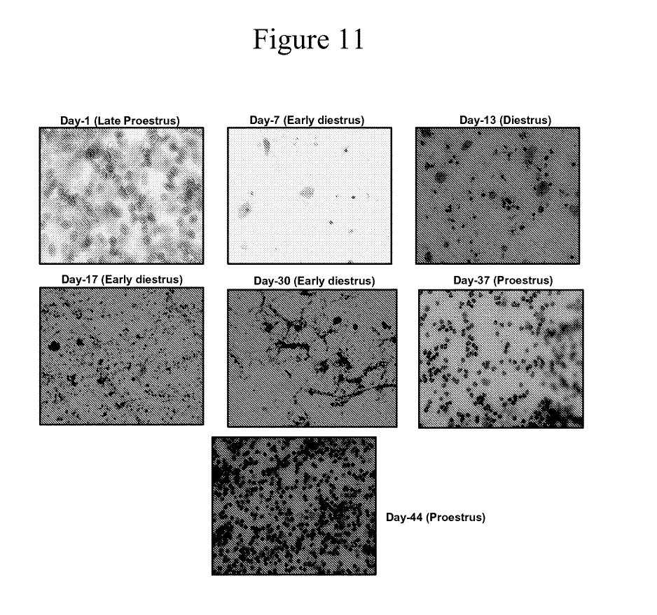

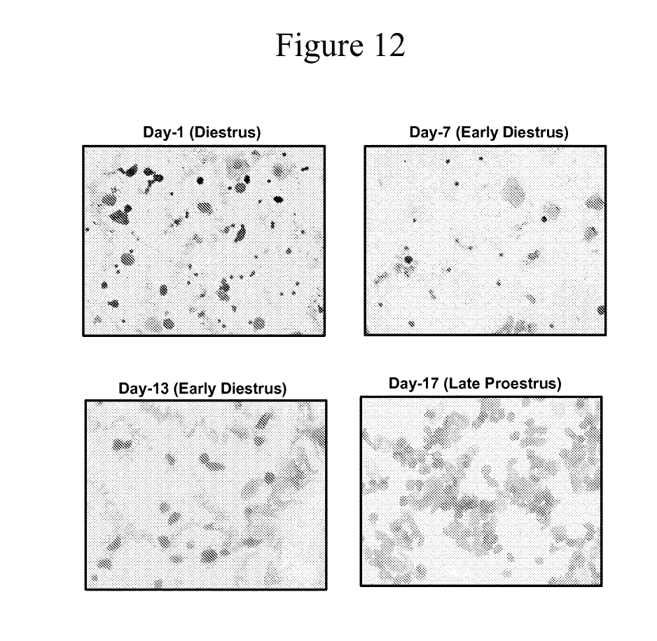

Rat vaginal cytology examination has been performed on the collected vaginal cells from the rats subcutaneously injected with the tamarin (FIG. 10), 55 LNG (FIGS. 11), and 96 LNG (FIG. 12) formulations. The representative images of the vaginal cells are shown in FIGS. 10-12. All of these cells are either from early diestrus, diestrus or proesterus stages. None of them show estrus stage.

### Example 8

### Pharmacokinetic Study

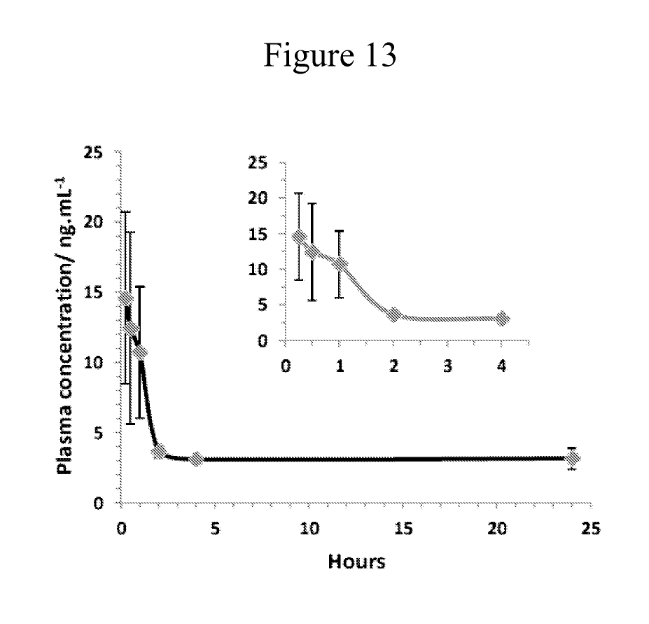

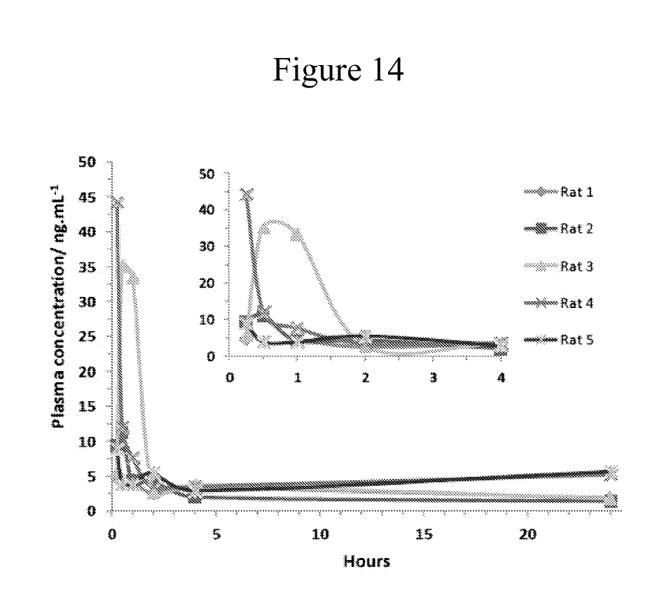

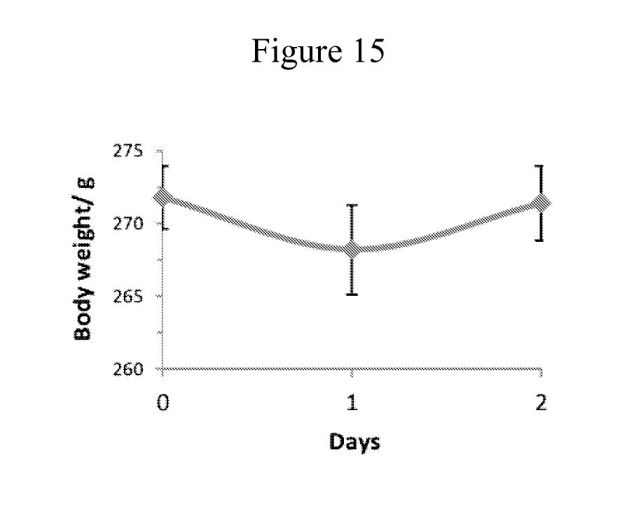

Tamarin-LNG formulation was injected subcutaneously into 5 female Sprague Dawley rats at a dose of 40 mg/kg. Blood samples were collected after the injection at 0, 0.25, 0.50, 1, 2, 4, and 24 h. Body weights were also measured at 0, 1 and 2 days after the injection. Plasma was isolated from all the blood samples and stored at −80° C. for PK studies using UFLC/MS/MS. FIG. 13 shows a burst release of LNG in the plasma at 14.6±6.1 ng·mL−1 at 15 min, but this burst is 5-6 times lower than that (97.8±4.8 ng·mL−1) with the 96 formulation. FIG. 14 shows the LNG concentration in the plasma measured in individual rats. FIG. 15 shows that the body weights of the rats did not change significantly for 2 days after the injection.

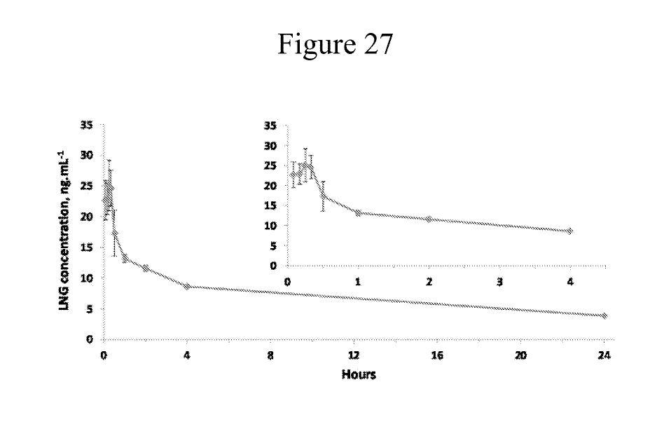

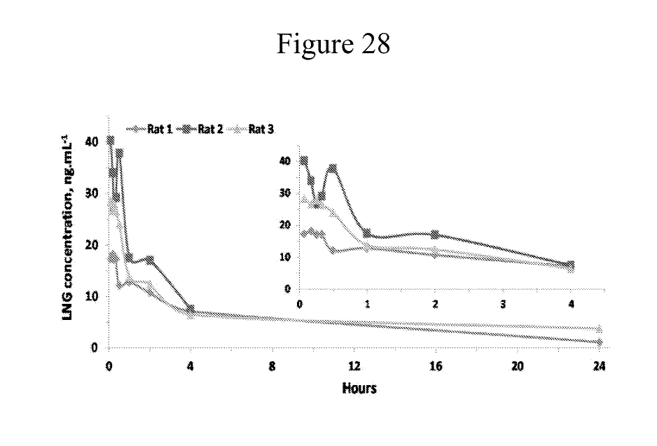

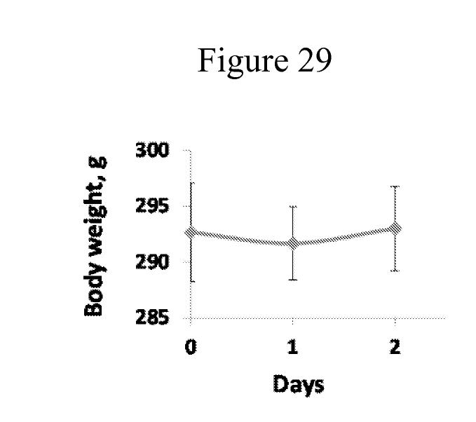

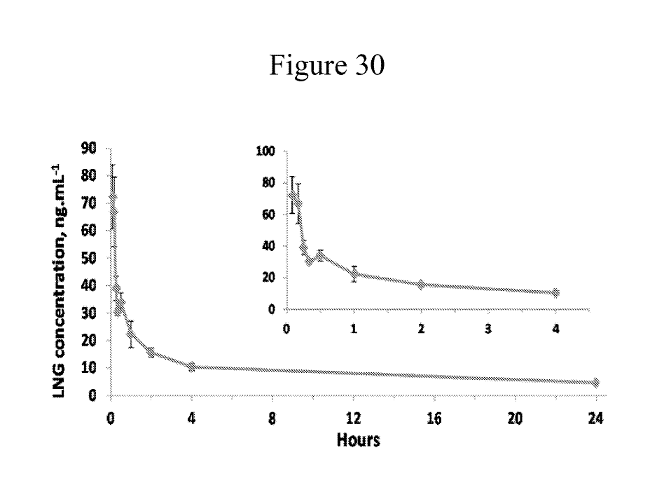

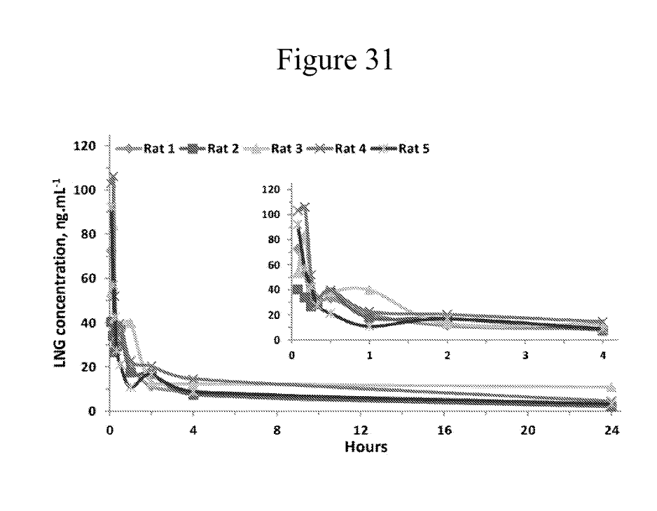

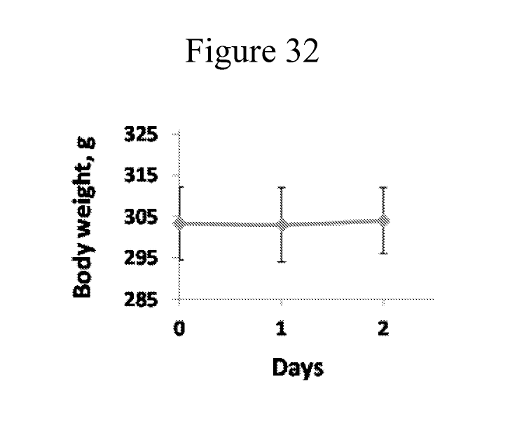

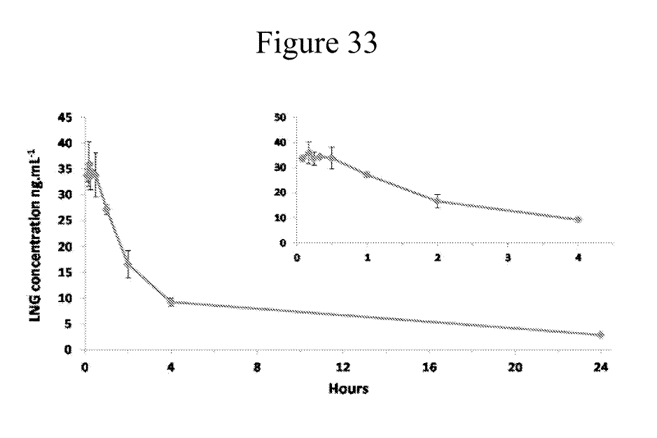

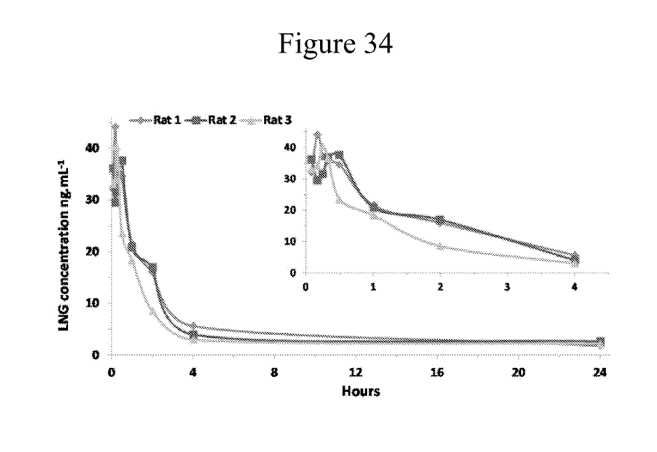

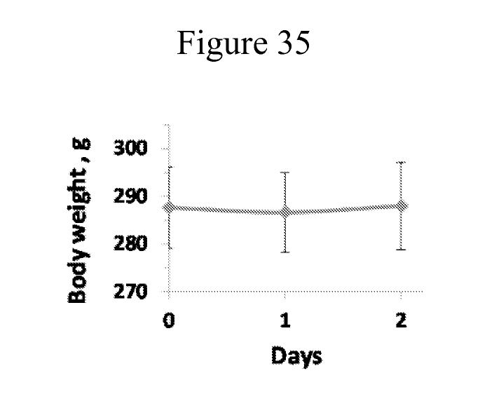

96.r, 96.zz and 64.a formulations (Table 6) were injected subcutaneously into separate groups of female Sprague Dawley rats at a dose of 40 mg/kg. Blood samples were collected after the injection at 0, 5, 10, 15, 20, 30, 60, 120 and 240 min and 24 h. Body weights were also measured at 0, 1 and 2 days after the injection. Plasma was isolated from all the blood samples and stored at −80° C. for PK studies using UFLC/MS/MS (FIGS. 27-35). Among the three formulations, 96.r showed the lowest burst of 25.08+4.11 ng/mL at 15 min after SubQ injection (FIGS. 27-35) whereas the initial burst was about 72.32+11.65 ng/mL (at 5 min) and 35.96+4.31 ng/mL (at 10 min) for 96.zz and 64.a formulations, respectively. Formulations with TEC as a secondary solvent (96.r and 64.a) had lower initial burst compared to the formulation with EA as a solvent (96.zz). FIGS. 29, 32 and 35 show that the body weight of the rats did not change during the two day studies.

400 μL of diluted tamarin-LNG formulation was injected subcutaneously into each of 5 female Sprague Dawley rats implanted with jugular catheters at a dose of 40 mg/kg. A 100 μL blood sample was drawn from each rat at selected time 0 (before injecting the formulation), 5, 10, 15, 20 and 30 min, and 1, 2, 4 and 24 h post the injection. Plasma was isolated from all the blood samples and stored at −80° C. The samples were analyzed by using LC/MS/MS for pharmacokinetic analysis. Diluted tamarin formulation had a Cmax=81.7±9.7 ng/mL (n=5), and formulation 96r had a Cmax=25±4 ng/mL (n=5) (FIG. 27).

TABLE 6
List of formulations tested for in vitro/in vivo LNG release studies.

| | PLGA | | | | |
|---|---|---|---|---|---|
| Formu- | 0.63, | PLA | PLA | LNG | Solvent |
| lation | wt % | 0.63, wt % | 0.47, wt % | wt % | 9:1, wt % |
| 96.r | 4 | 6 | 16 | 4 | NMP/TEC, 72% |
| 96.zz | 4 | 6 | 16 | 4 | NMP/EA, 72% |
| 64.a | 4 | 10 | 8 | 4 | NMP/TEC, 74% |

### Example 9

### Method Development for UPLC/MS/MS Analysis of Mixture of LNG and LNG-B

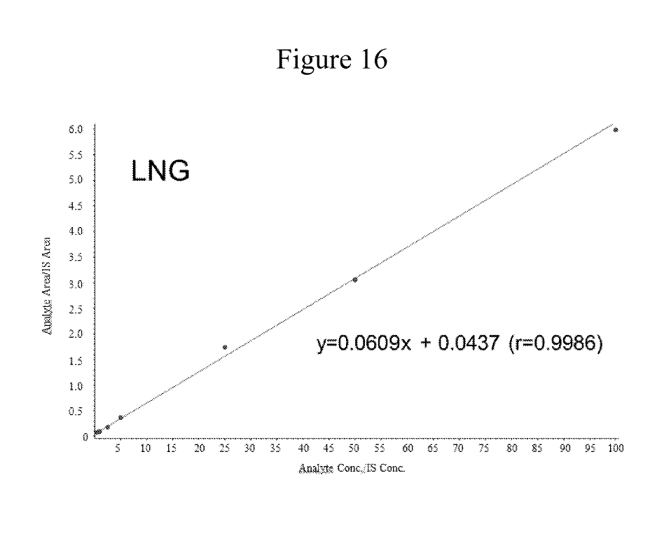

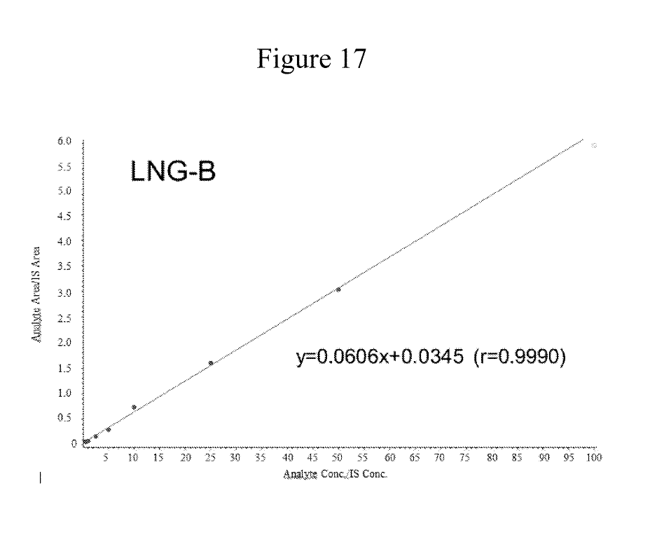

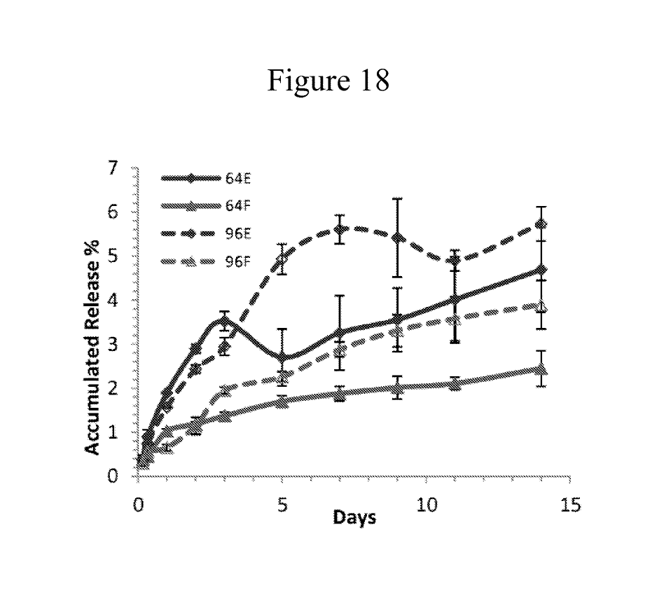

LNG and LNG-B at a concentration of 0.5 (this concentration was not used for LNG-B), 1, 2.5, 5, 10, 25, 50 or 100 ng·mL−1 each were spiked together into 90 μL of rat plasma along with LNG-D6 internal standard. LNG and LNG-B were extracted from the plasma using mixture of hexane and ethyl acetate at 70:30 v:v, vacuum dried, and dissolved in mixture of water and acetonitrile at 80:20 v:v for UFLC/MS/MS analysis. FIGS. 16 and 17 shows the standard graphs for LNG and LNG-B obtained on the ABSceix API 4500 mass spectrometer.

### Example 10

### In Vitro Accelerated Release Method Development

96 and 64 (tamarin) formulations and two in vitro release conditions listed in Table 7 were used for the accelerated in vitro release method development. Each gel was formed by injecting LNG-containing polymeric solution in a Teflon mold and then immersed in 400 mL release medium in a flask. The in vitro release was conducted in a shaker at 50° C. for two weeks. At selected time points, the entire release media were taken out and replaced immediately with fresh release media. The collected sample solutions from 96 and tamarin formulations were analyzed for drug content using LC/MS/MS. In vitro LNG release rates were expressed as percentage cumulative amount as a function of time.

FIG. 18 shows that in PBS (pH 11), the presence of surfactant tween 20 caused more LNG release than surfactant poloxamer 407 from both the tamarin and 96 formulations. More LNG was released from the 96 formulation than the tamarin formulation in both E and F conditions in general. pH 11 appears to cause slower LNG release kinetics than pH 9 in PBS containing 25% ethanol and 2 g (0.5%) tween 20.

TABLE 7
Conditions used for accelerated in vitro release method development.

| | PBS | Ethanol | Tween 20 | Poloxamer 407 | |
|---|---|---|---|---|---|
| Conditions | (mL) | (mL) | (g) | (g) | pH |
| E | 300 | 100 | 2 | | 11 |
| F | 300 | 100 | | 2 | 11 |

### Example 11

### In Vitro Accelerated Release Studies (Formulation Optimization)

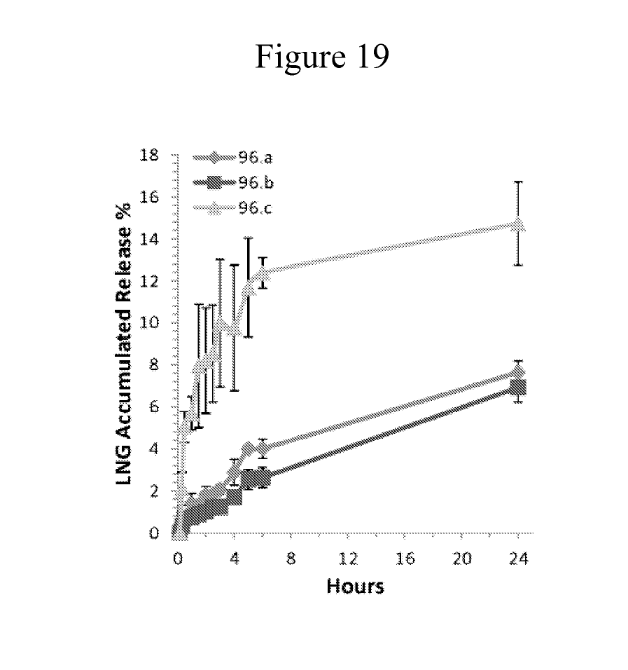

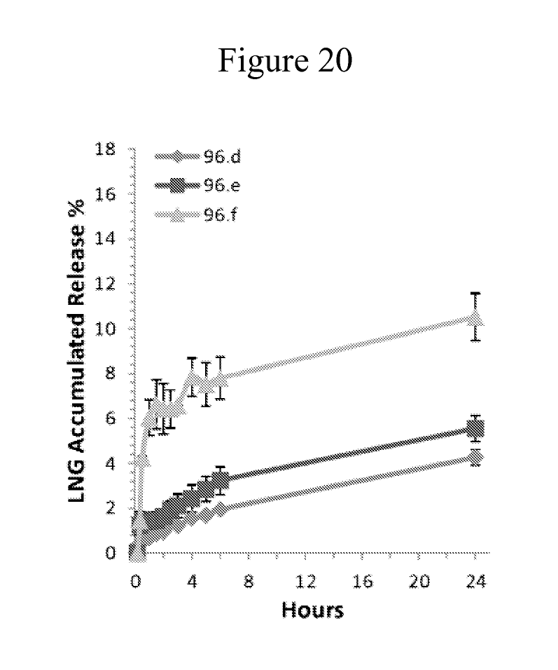

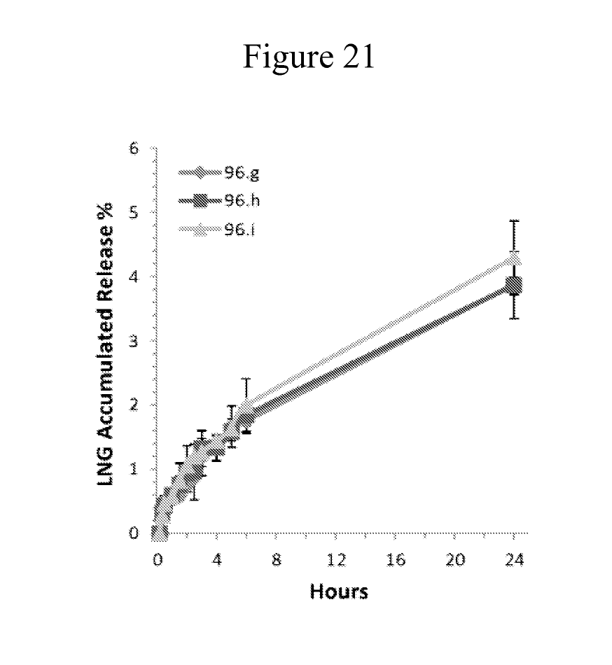

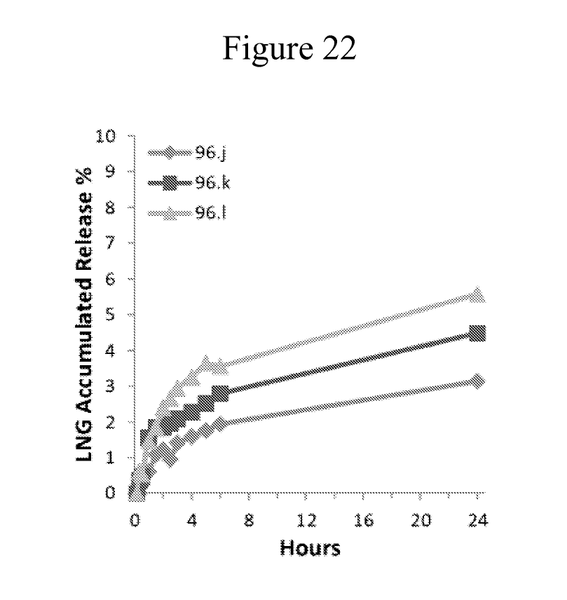

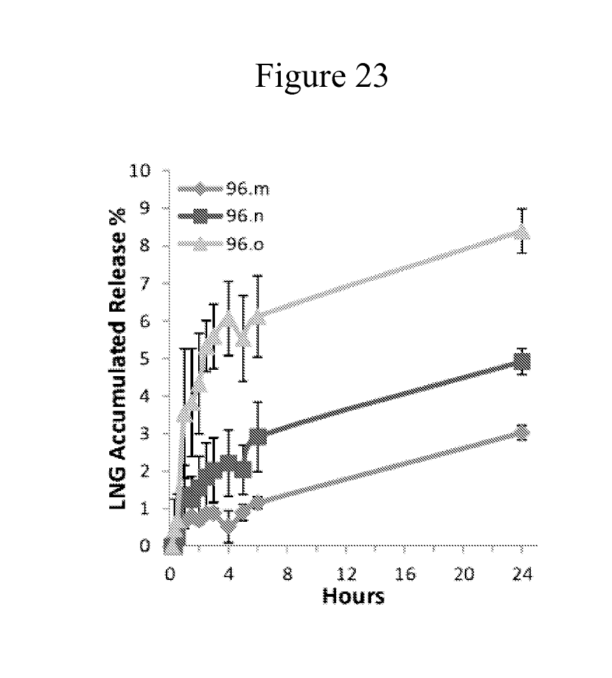

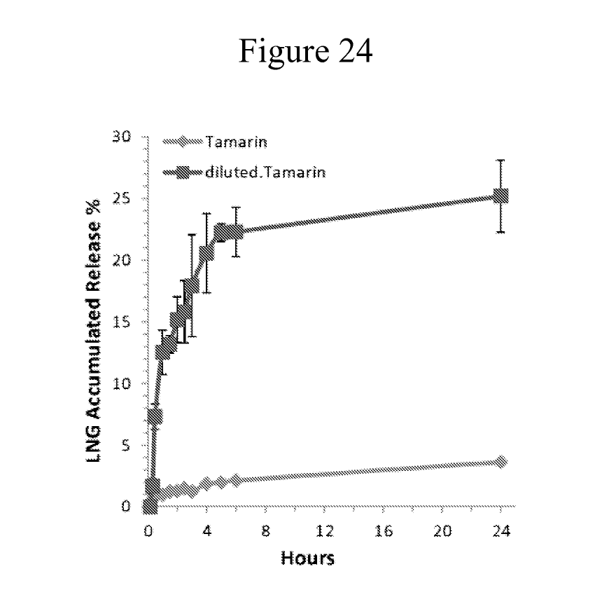

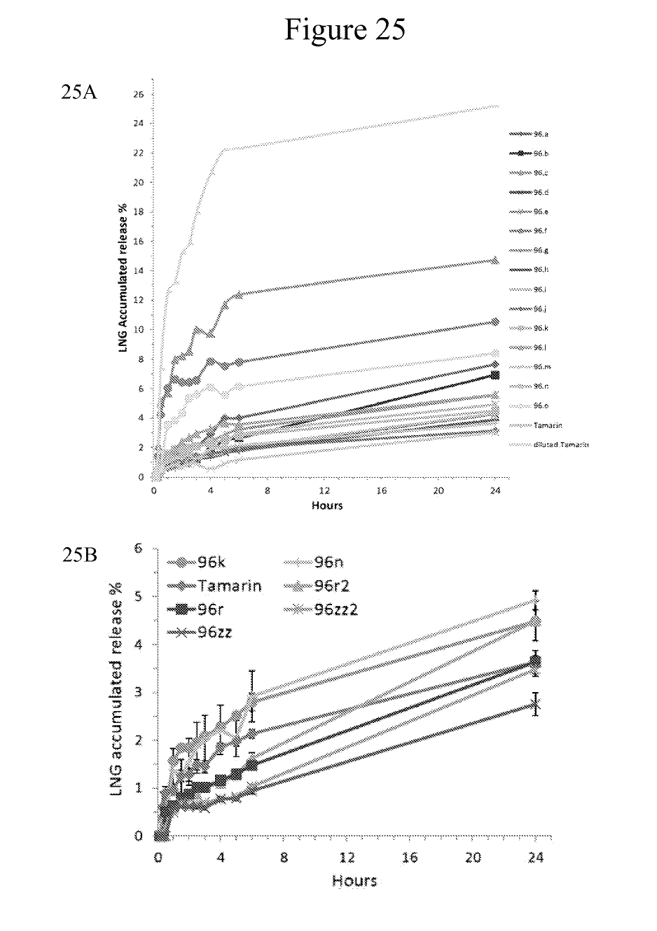

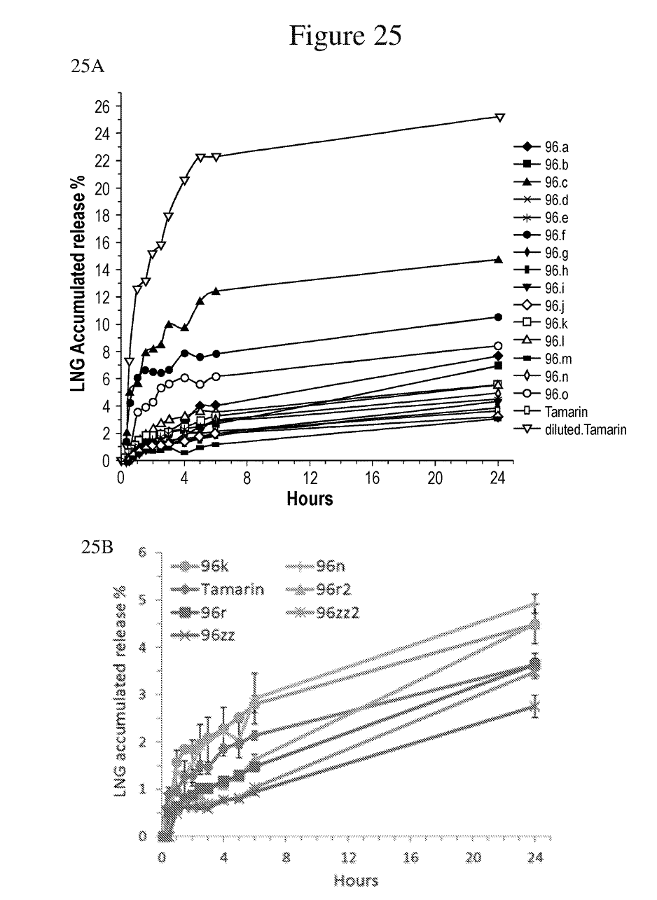

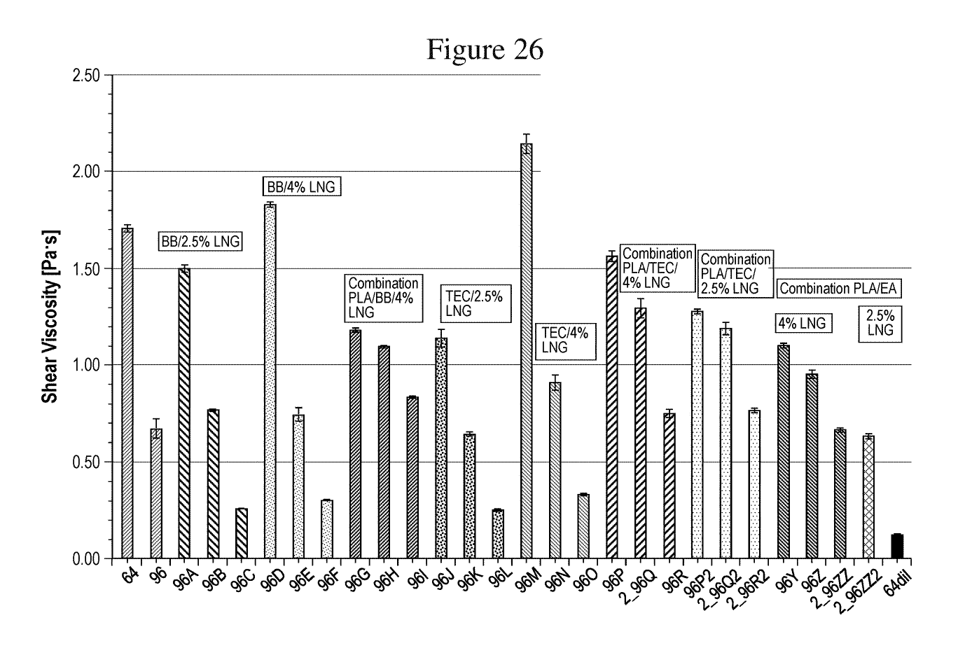

Formulations listed in Table 8 were prepared by altering the polymer intrinsic viscosity and concentration, and drug concentration based on the 96 formulation. Based on the previous in vitro accelerated release method development results, a release medium containing PBS (pH 9), 25% ethanol and 0.5% tween 20 and a temperature of 50° C. was chosen for conducting in vitro accelerated release study of the new formulations for initial burst study. Release samples were collected at selected time points, 10, 20 and 30 min and 1, 1.5, 2, 2.5, 3, 4, 5, 6 and 24 h. FIGS. 19-23 show the LNG accumulated release from different formulations containing NMP/BB and NMP/TEC as solvent systems, respectively. FIG. 24 shows the LNG accumulated release from tamarin and diluted tamarin formulations. FIGS. 25A, 25B, 25C, and 25D show the summary of LNG accumulated release from formulations listed in Table 8. Formulations 96k, tamarin, 96n, 96r, 96r2, 96zz and 96zz2 were injectable through 23 gauge needles, have low initial burst and slow in vitro LNG release in a comparable way to tamarin. LNG is released from formulations containing 2.5 wt % LNG more than or the same as those containing 4 wt % LNG.

TABLE 8
Formulations tested for accelerated release studies.

| | PLGA | | | | |
|---|---|---|---|---|---|
| | wt % | PLA | | | |
| | (50:50) | wt % | PLA wt % | LNG | Solvents wt % |
| Sample ID | (IV 0.63) | (IV 0.47) | (IV 0.63) | wt % | (9:1 w:w) |
| 96 | 4.1 | 20.3 | | 2.5 | 73.1% NMP/BB |
| Tamarin | 4.7 | | 18.8 | 6 | 70.5% NMP/TEC |
| (64) | | | | | |
| diluted. | 1.8 | | 7.5 | 2.4 | 88.3% NMP/TEC |
| Tamarin | | | | | |
| 96.a | 4 | | 20 | 2.5 | 73.5% NMP/BB |
| 96.b | 4 | | 16 | 2.5 | 77.5% NMP/BB |
| 96.c | 4 | | 12 | 2.5 | 81.5% NMP/BB |
| 96.d | 4 | | 20 | 4 | 72% NMP/BB |
| 96.e | 4 | | 16 | 4 | 76% NMP/BB |
| 96.f | 4 | | 12 | 4 | 80% NMP/BB |
| 96.g | 4 | 4 | 16 | 4 | 72% NMP/BB |
| 96.h | 4 | 8 | 12 | 4 | 72% NMP/BB |
| 96.i | 4 | 16 | 4 | 4 | 72% NMP/BB |
| 96.j | 4 | | 20 | 2.5 | 73.5% NMP/TEC |
| 96.k | 4 | | 16 | 2.5 | 77.5% NMP/TEC |
| 96.l | 4 | | 12 | 2.5 | 81.5% NMP/TEC |
| 96.m | 4 | | 20 | 4 | 72% NMP/TEC |
| 96.n | 4 | | 16 | 4 | 76% NMP/TEC |
| 96.o | 4 | | 12 | 4 | 80% NMP/TEC |
| 96.p | 4 | 4 | 16 | 4 | 72 NMP/TEC |
| 96.q | 4 | 8 | 12 | 4 | 72 NMP/TEC |
| 96.r | 4 | 16 | 4 | 4 | 72 NMP/TEC |
| 96.p2 | 4 | 4 | 16 | 2.5 | 73.5 NMP/TEC |
| 96.q2 | 4 | 8 | 12 | 2.5 | 73.5 NMP/TEC |
| 96.r2 | 4 | 16 | 4 | 2.5 | 73.5 NMP/TEC |
| 96.s | 4 | | 20 | 2.5 | 73.1 NMP/EA |
| 96.t | 4 | | 16 | 2.5 | 77.5 NMP/EA |
| 96.u | 4 | | 12 | 2.5 | 81.5 NMP/EA |
| 96.v | 4 | | 20 | 4 | 76 NMP/EA |
| 96.w | 4 | | 16 | 4 | 76 NMP/EA |
| 96.x | 4 | | 12 | 4 | 80 NMP/EA |
| 96.y | 4 | 4 | 16 | 4 | 72 NMP/EA |
| 96.z | 4 | 8 | 12 | 4 | 72 NMP/EA |
| 96.zz | 4 | 16 | 4 | 4 | 72 NMP/EA |
| 96.y2 | 4 | 4 | 16 | 2.5 | 73.5 NMP/EA |
| 96.z2 | 4 | 8 | 12 | 2.5 | 73.5 NMP/EA |
| 96.zz2 | 4 | 16 | 4 | 2.5 | 73.5 NMP/EA |

### Example 12

### Injectability

The injectability of the new formulations through different size gauge needles: 18, 21, and 23 G was tested and the results were listed in Table 9 according to the scale 0-5 with 0 indicating not injectable and 5 indicating very easily injectable.

TABLE 9
Injectability of the formulations through 18G, 21G and 23G needles.

| Sample ID | 23 G | 21 G | 18 G |
|---|---|---|---|
| 96 | 4 | 4 | 5 |
| 96.a | 2 | 3 | 4 |
| 96.b | 4 | 4 | 5 |
| 96.c | 4 | 5 | 5 |
| 96.d | 2 | 3 | 4 |
| 96.e | 4 | 4 | 5 |
| 96.f | 4 | 5 | 5 |
| 96.g | 2 | 3 | 4 |
| 96.h | 2 | 3 | 4 |
| 96.i | 3 | 4 | 4 |
| 96.j | 2 | 3 | 4 |
| 96.k | 4 | 4 | 5 |
| 96.l | 4 | 5 | 5 |
| 96.m | 2 | 3 | 4 |
| 96.n | 4 | 4 | 5 |
| 96.o | 4 | 5 | 5 |
| Tamarin | 2 | 2 | 4 |
| diluted.Tamarin | 5 | 5 | 5 |
| 96p | 2 | 3 | 4 |
| 96q | 2 | 3 | 4 |
| 96r | 3 | 4 | 5 |
| 96p2 | 2 | 3 | 4 |
| 96q2 | 2 | 3 | 4 |
| 96r2 | 3 | 4 | 5 |
| 96zz | 4 | 4 | 5 |
| 96zz2 | 4 | 4 | 5 |

The injectability was rated on a scale from 0-5 (0-Not injectable; 1-Very Hard; 5-Very Easy).

Among all the studied formulations, when the total polymer concentration (PLGA wt % +PLA wt %) was decreased from 24% to 16%, the viscosity decreased, the injectability was better, and LNG was released more with higher burst (FIGS. 19-24, 25A, 25B, 25C, and 25D). Also, it was observed that formulations containing TEC as a solvent released less LNG compared to those formulations containing BB as solvent (FIGS. 19-23). By keeping a balance between injectability and LNG release, the formulations containing a total polymer concentration of 20% (96b, e, k, and n) with viscosity below 1 Pa·s can serve as better candidates in terms of injectability and low burst release. Among these four formulations, the 96k and 96n formulations showed similar release profiles for LNG but better injectability when compared to the tamarin formulation.

What is claimed is:

1. An injectable polymer matrix drug delivery system comprising:
a) a biodegradable polymer selected from the group consisting of polyester, poly(lactic-co-glycolic acid), poly(lactic acid), poly(ε-caprolactone), poly(ethylene glycol-block-lactic acid), poly(alkylcyanoacrylate), polyanhydride, poly(bis(p-carboxyphenoxy) propane-sebacic acid), polyorthoester, polyphosphoester, polyphosphazene, polyurethane, and poly(amino acid), or combinations thereof;
b) a solvent or a combination of solvents; and
c) an active pharmaceutical ingredient.

2. The drug delivery system of claim 1, wherein the biodegradable polymer is selected from poly(lactic-co-glycolic acid), poly(lactic acid), and poly(ε-caprolactone), or combinations thereof.

3. The drug delivery system of claim 1, wherein the solvent is selected from N-methyl-2-pyrrolidone (NMP), benzyl benzoate (BB), benzyl alcohol (BA), triethyl citrate (TEC), acetyl triethyl citrate (ATEC), ethyl acetate (EA), and acetyl tributyl citrate (ATBC), or combinations thereof.

4. The drug delivery system of claim 1, wherein the solvent is selected from N-methyl-2-pyrrolidone (NMP), benzyl benzoate (BB), benzyl alcohol (BA), triethyl citrate (TEC), acetyl triethyl citrate (ATEC), and ethyl acetate (EA), or combinations thereof.

5. The drug delivery system of claim 1, wherein the biodegradable polymer is selected from poly(L-lactic acid) and poly(D,L-lactic acid), or combinations thereof.

6. The drug delivery system of claim 1, wherein the active pharmaceutical ingredient is an anti-inflammatory agent, an antibacterial agent, an antiparasitic agent, an antifungal agent, an antiviral agent, an anti-neoplastic agent, an analgesic agent, an opioid, a drug for the treatment of arthritis, a drug for the treatment of rheumatoid arthritis, an antibody, a monoclonal antibody, a protein drug, a peptide drug, a gene, an enzyme, an antibiotic, a nucleic acid, a DNA, a RNA, a receptor, an antipsychotic, an anesthetic, a vaccine, a central nervous system agent, a growth factor, a hormone, an antihistamine, an osteoinductive agent, a cardiovascular agent, an anti-ulcer agent, a bronchodilator, a vasodilator, a birth control agent, a fertility enhancing agent,interferon alpha, a hormone, a growth hormone, an osteoporosis drug, parathyroid hormone, an obesity drug, a psychiatric drug, an anti-diabetes drug, a treatment for female infertility, an AIDS treatment, a hepatitis drug, a multiple sclerosis drug, a migraine headache drug, an allergic reaction treatment, interferon consensus, interleukin, erythropoietin, granulocyte-colony stimulating factor (GCSF), stem cell factor (SCI), leptin (OB protein), interferon (alpha, beta, gamma), ciprofloxacin, amoxycillin, lactobacillus, cefotaxime, levofloxacin, cefipime, mebendazole, ampicillin, lactobacillus, cloxacillin, norfloxacin, tinidazole, cefpodoxime, proxctil, azithromycin, gatifloxacin, roxithromycin, cephalosporin, anti-thrombogenics, aspirin, ticlopidine, sulfinpyrazone, heparin, warfarin, growth factors, differentiation factors, hepatocyte stimulating factor, plasmacytoma growth factor, brain derived neurotrophic factor (BDNF), glial derived neurotrophic factor (GDNF), neurotrophic factor 3 (NT3), fibroblast growth factor (FGF), transforming growth factor (TGF), platelet transforming growth factor, milk growth factor, endothelial growth factors (EGF), endothelial cell-derived growth factors (ECDGF), alpha-endothelial growth factors, beta-endothelial growth factor, neurotrophic growth factor, nerve growth factor (NGF), vascular endothelial growth factor (VEGF), 4-1 BB receptor (4-1BBR), TRAIL (TNF-related apoptosis inducing ligand), artemin (GFRalpha3-RET ligand), BCA-1 (B cell-attracting chemokine1), B lymphocyte chemoattractant (BLC), B cell maturation protein (BCMA), brain-derived neurotrophic factor (BDNF), bone growth factor such as osteoprotegerin (OPG), bone-derived growth factor, megakaryocyte derived growth factor (MGDF), keratinocyte growth factor (KGF), thrombopoietin, platelet-derived growth factor (PGDF), megakaryocyte derived growth factor (MGDF), keratinocyte growth factor (KGF), platelet-derived growth factor (PGDF), bone morphogenetic protein 2 (BMP2), BRAK, C-10, Cardiotrophin 1 (CT1), CCR8, anti-inflammatory: paracetamol, salsalate, diflunisal, mefenamic acid, diclofenac, piroxicam, ketoprofen, dipyrone, acetylsalicylic acid, antimicrobials amoxicillin, ampicillin, cephalosporins, erythromycin, tetracyclines, penicillins, trimethprim-sulfamethoxazole, quniolones, amoxicillin, clavulanatf, azithromycin, clarithromycin, anti-cancer drugs aliteretinoin, altertamine, anastrozole, azathioprine, bicalutamide, busulfan, capecitabine, carboplatin, cisplatin, cyclophosphamide, cytarabine, doxorubicin, epirubicin, etoposide, exemestane, vincristine, vinorelbine, hormones, thyroid stimulating hormone (TSH), sex hormone binding globulin (SHBG), prolactin, luteotropic hormone (LTH), lactogenic hormone, parathyroid hormone (PTH), melanin concentrating hormone (MCH), luteinizing hormone (LHb), growth hormone (HGH), follicle stimulating hormone (FSHb), haloperidol, indomethacin, doxorubicin, epirubicin, amphotericin B, Taxol, cyclophosphamide, cisplatin, methotrexate, pyrene, amphotericin B, anti-dyskinesia agents, Alzheimer vaccine, antiparkinson agents, ions, edetic acid, nutrients, glucocorticoids, heparin, anticoagulation agents, anti-virus agents, anti-HIV agents, polyamine, histamine and derivatives thereof, cystineamine and derivatives thereof, diphenhydramine and derivatives, orphenadrine and derivatives, muscarinic antagonist, phenoxybenzamine and derivatives thereof, protein A, streptavidin, amino acid, beta-galactosidase, methylene blue, protein kinases, beta-amyloid, lipopolysaccharides, eukaryotic initiation factor-4G, tumor necrosis factor (TNF), tumor necrosis factor-binding protein (TNF-bp), interleukin-1 (to 18) receptor antagonist (IL-Ira), granulocyte macrophage colony stimulating factor (GM-CSF), novel erythropoiesis stimulating protein (NESP), thrombopoietin, tissue plasminogen activator (TPA), urokinase, streptokinase, kallikrein, insulin, steroid, acetylsalicylic acid, acetaminophen, analgesic, anti-tumor preparation, anti-cancer preparation, anti-proliferative preparation or pro-apoptotic preparation.

7. The drug delivery system of claim 1, wherein the active pharmaceutical ingredient is an anti-inflammatory agent, an antibacterial agent, an antiparasitic agent, an antifungal agent, an antiviral agent, an anti-neoplastic agent, an analgesic agent, an opioid, a drug for the treatment of arthritis, a drug for the treatment of rheumatoid arthritis, an antibody, a monoclonal antibody, a protein drug, a peptide drug, an antipsychotic, an anesthetic, a vaccine, a central nervous system agent, a growth factor, a hormone, an antihistamine, an osteoinductive agent, a cardiovascular agent, an anti-ulcer agent, a bronchodilator, a vasodilator, a birth control agent, a fertility enhancing agent, interferon alpha, a growth hormone, an osteoporosis drug, parathyroid hormone, an obesity drug, a psychiatric drug, an anti-diabetes drug, a treatment for female infertility, an AIDS treatment, a hepatitis drug, a multiple sclerosis drug, a migraine headache drug, or an allergic reaction treatment.

8. The drug delivery system of claim 1, wherein active pharmaceutical ingredient is a birth control agent.

9. The drug delivery system of claim 8, wherein the birth control agent is human progestogen, progesterone, norethisterone, ethynodiol diacetate, norethynodrel, dienogest, lynestrenol, medroxyprogesteroneacetate, megestroneacetate, levonorgestrel, levonorgestrel butanoate, norgestrel, desogestrel, gestodene, norgestimate, etonorgestrel, drospirenone, dienogest, or ethinylestradiol, or combinations thereof.

10. The drug delivery system of claim 8, wherein the birth control agent is levonorgestrel or levonorgestrel butanoate.

11. The drug delivery system of claim 1, wherein the solvent is selected from N-methyl-2-pyrrolidone (NMP), benzyl benzoate (BB), benzyl alcohol (BA), triethyl citrate (TEC), acetyl triethyl citrate (ATEC), and ethyl acetate (EA), or combinations thereof, the biodegradable polymer is selected from poly(L-lactic acid) and poly(D,L-lactic acid), or combinations thereof, and the active pharmaceutical ingredient is human progestogen, progesterone, norethisterone, ethynodiol diacetate, norethynodrel, dienogest, lynestrenol, medroxyprogesteroneacetate, megestroneacetate, levonorgestrel or levonorgestrel butanoate, norgestrel, desogestrel, gestodene, norgestimate, etonorgestrel, drospirenone, dienogest, or ethinylestradiol, or combinations thereof.

12. The drug delivery system of claim 11, wherein the active pharmaceutical ingredient is levonorgestrel or levonorgestrel butanoate.

13. The drug delivery system of claim 1, wherein the solvent combination is NMP and TEC; NMP and ATEC; NMP and ATBC; NMP and BB; NMP and BA; NMP and EA;
TEC and BB; ATEC and BB; ATBC and BB; TEC and BA; ATEC and BA; ATBC and BA;
TEC and EA; ATEC and EA; ATBC and EA; NMP, TEC and BB; NMP, ATEC and BB; NMP, ATBC and BB; NMP, TEC and BA; NMP, ATEC and BA; NMP, ATBC and BA; NMP, TEC and EA; NMP, ATEC and EA; NMP, ATBC and EA; TEC, BB and EA; ATEC, BB and EA; ATBC, BB and EA; TEC, BA and EA; ATEC, BA and EA; or ATBC, BA and EA.

14. The drug delivery system of claim 1, wherein the solvent combination is NMP and TEC, NMP and ATEC, NMP and BB, or NMP and BA.

15. The drug delivery system of claim 1, wherein the system comprises the polymer in about 0-50% by weight, the solvent in about 50-95% by weight, and the pharmaceutical ingredient in about 0.1-30% by weight.

16. A method of inducing amenorrhea, the method comprising administering to a subject in need thereof the injectable polymer matrix drug delivery system of claim 1.

17. A method of reducing or inhibiting spermatogenesis, the method comprising administering to a subject in need thereof the injectable polymer matrix drug delivery system of claim 1.

18. A method of minimizing uterine bleeding, the method comprising administering to a subject in need thereof the injectable polymer matrix drug delivery system of claim 1.

19. A method of minimizing estrus, the method comprising administering to a subject in need thereof the injectable polymer matrix drug delivery system of claim 1.

20. The method of claim 16, wherein upon administration to the subject in need thereof, the active pharmaceutical ingredient is continuously released at a rate according to a zero order reaction from about 0 months to about 18 months.

21. The method of claim 16, wherein upon administration to the subject in need thereof, the active pharmaceutical ingredient is released for at least 3 months.

22. The method of claim 16, wherein the polymer matrix is injected through a needle of about 18-gauge to about 26-gauge.

23. The method of claim 16, wherein the polymer matrix is injected through a needle of about 23-gauge to about 26-gauge.

24. The method of claim 16, wherein the system is formulated for subcutaneous injection or intramuscular injection.

25. The method of claim 16, wherein the system forms a semi-solid or solid depot at the injection site.

26. A method of forming a polymer matrix drug delivery system of claim 1 comprising:
a) adding an active pharmaceutical ingredient to a solvent or a combination of solvents;
b) dissolving or dispersing the active pharmaceutical ingredient;
c) adding the dissolved or dispersed active pharmaceutical solution to a biodegradable polymer selected from the group consisting of poly(lactic-co-glycolic acid), poly(lactic acid), poly(ε-caprolactone), poly(ethylene glycol-block-lactic acid), poly(alkylcyanoacrylate), polyanhydride, poly(bis(p-carboxyphenoxy) propane-sebacic acid), polyorthoester, polyphosphoester, polyphosphazene, polyurethane, and poly(amino acid), or combinations thereof; and
d) mixing the dissolved or dispersed active pharmaceutical ingredient and biodegradable polymer solution to homogeneity;
such that the polymer matrix drug delivery system is formed.
